(19) United States
(12) Patent Application Publication
(10) Pub. No.: US 2015/0051270 A1
(43) Pub. Date: Feb. 19, 2015

(54) Novel Cyclic Phenoxy Compounds and Improved Treatments for Cardiac and Cardiovascular Disease

(71) Applicant: University of Nottingham, Nottingham, Notthinghamshire (GB)

(72) Inventors: Shailesh Mistry, Nottingham (GB); Etíenne Daras, Nottingham (GB); Christophe Fromont, Nottingham (GB); Gopal Jadhav, Nottingham (GB); Peter Martin Fischer, Nottingham (GB); Barrie Kellam, Nottingham (GB); Stephen John Hill, Nottingham (GB); Jillian Glenda Baker, Nottingham (GB)

(21) Appl. No.: 14/379,203

(22) PCT Filed: Feb. 15, 2013
(86) PCT No.: PCT/GB2013/050357

§ 371 Date: Aug. 15, 2014

(30) Foreign Application Priority Data
Feb. 15, 2012 (GB) 1202613.4

(51) Int. Cl. C07D 319/20; C07D 311/32; C07D 307/85; C07D 311/18

(52) U.S. Cl. CPC C07D 319/20; C07D 311/18; C07D 311/32; C07D 307/85
USPC 514/452; 514/456; 514/469; 549/362; 549/366; 549/399; 549/403; 549/467

(57) ABSTRACT

A compound of formula I, and its pharmaceutically acceptable salt or salts and physiologically hydrolysable derivatives in free form or salt form:

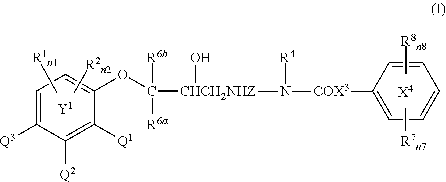

wherein
either Q¹, CR^{6a} and optionally R^{6b} together form a cyclic moiety wherein:
   Q¹ is selected from C₁₋₂ alkylene, C₁₋₂ alkenylene, OC₁ alkylene and OC₁ alkenylene moieties optionally substituted by oxo;
   R^{6a} is a single bond and R^{6b} is H; or
   R^{6a} and R^{6b} together form a double bond; and
   Q² and Q³ are independently selected from H, R¹ and R²;
or Q² and Q³ together form a cyclic moiety in which one of Q² and Q³ is a cyclic moiety selected from OC₁ alkylene and OC₁ alkenylene moieties optionally substituted by oxo or a group R⁵ as here in below defined for R² and the other of Q² and Q³ is a cyclic moiety selected from C₁₋₂ alkylene, C₁₋₂ alkenylene and OC₁ alkylene optionally substituted by oxo;
   R^{6a} and R^{6b} are each H or a cyclic moiety as defined above; and
   Q¹ is selected from H, R¹ and R² and a cyclic moiety as defined above;
and R¹⁻⁴ are H or substituents;
Z is selected from linear C₂₋₃ alkylene;
X³ is NH;
R⁷⁻⁹ are H or substituents; their preparation and novel intermediates, compositions thereof and their use in the prevention or treatment of cardiac and cardiovascular disease and methods for the treatment thereof.

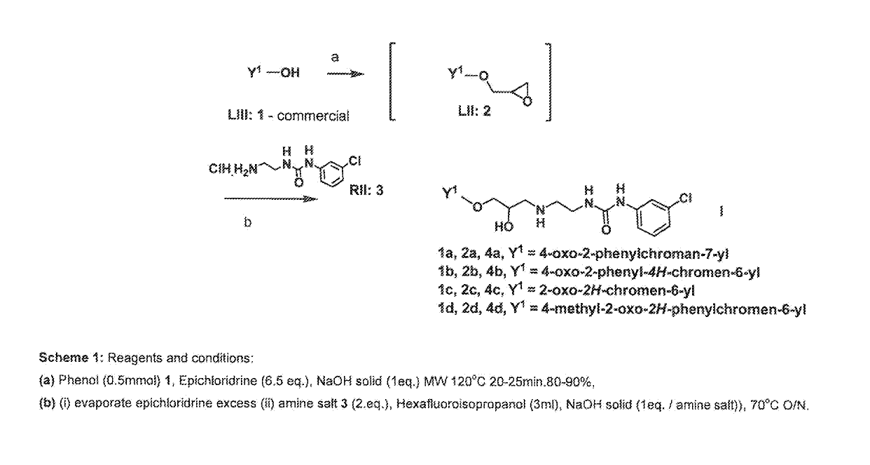

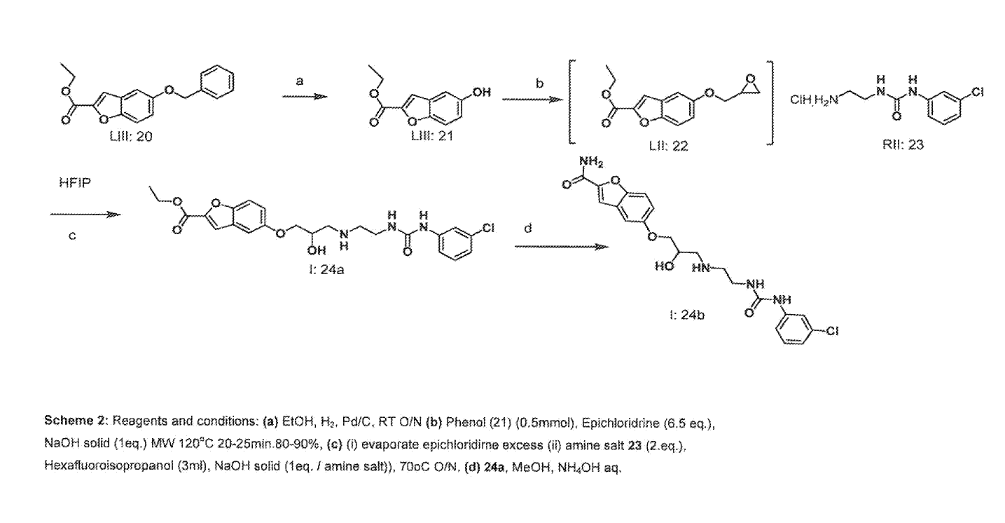

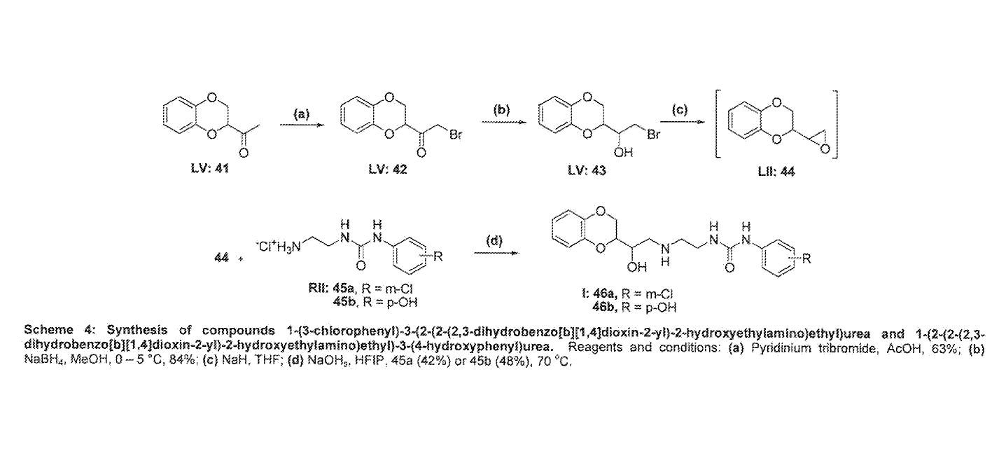

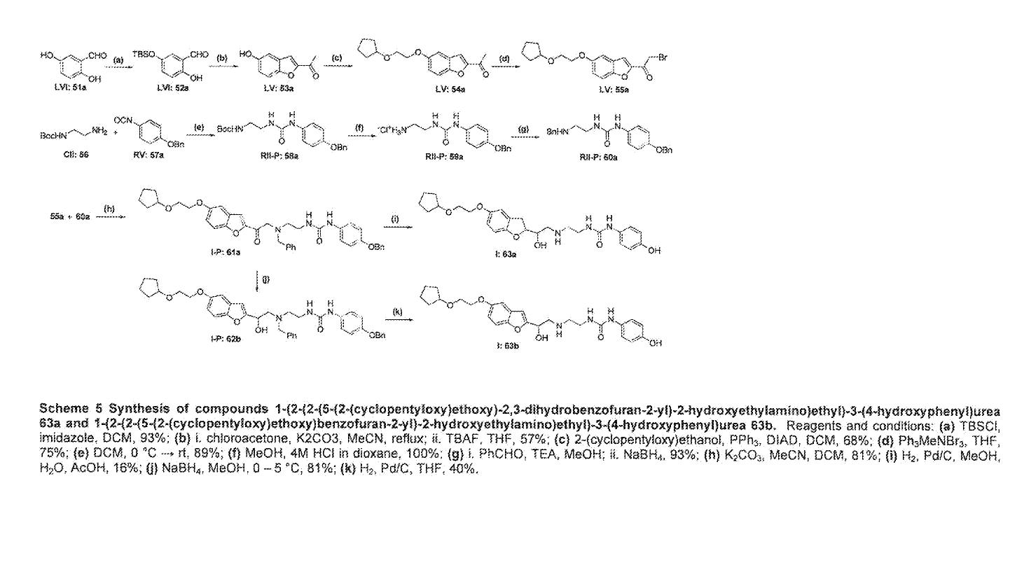

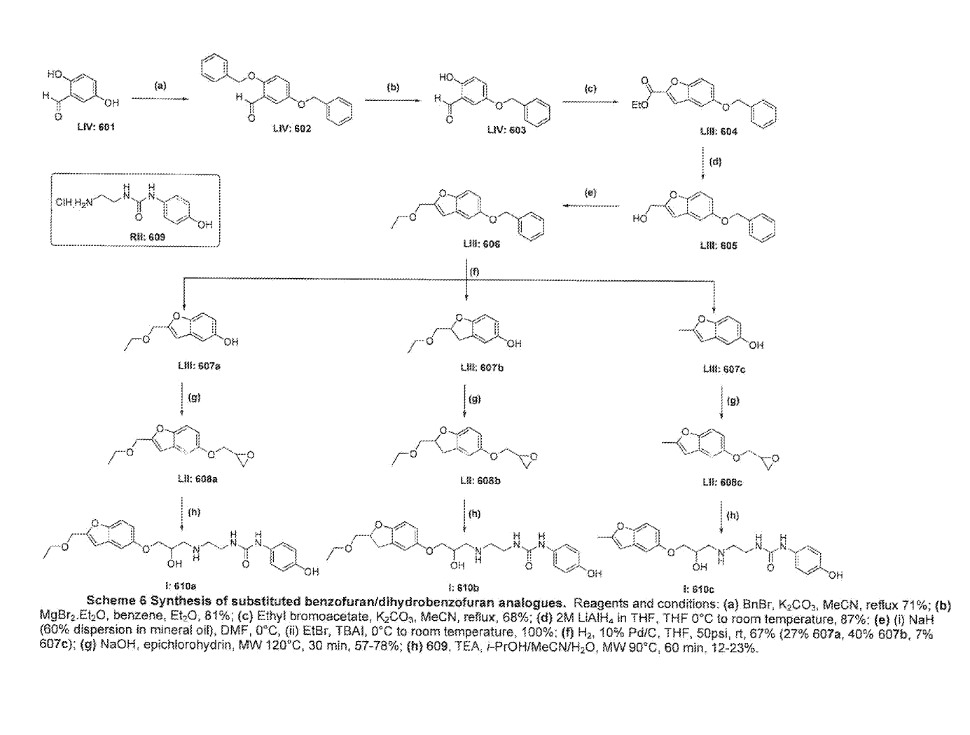

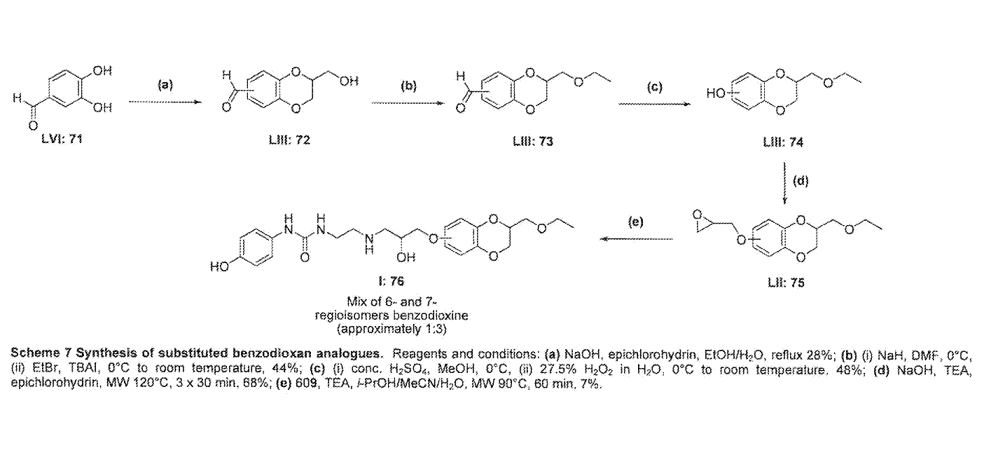

This invention relates to novel compounds and their preparation and use in treating cardiac and cardiovascular disease.

### BACKGROUND

β-adrenoceptor antagonists (β-blockers) are one of the most important therapies in the management of symptoms of, and for prolonging life in, cardiovascular disorders e.g. ischaemic heart disease and cardiac arrhythmias. They work by blocking the β1-adrenoceptors in the heart and thus prevent the endogenous hormones adrenaline and noradrenaline from increasing heart rate and force of contraction, β-blockers are also widely used in the management of hypertension, and (although the mechanism of action is not yet understood) they prolong life in patients with heart failure.

However, they are contraindicated in patients with respiratory disease (especially asthma and chronic obstructive pulmonary disease, COPD) because antagonism of the β2-adrenoceptors in the airways, results in bronchoconstriction and a loss of action of the important β2-agonist bronchodilators. Thus, currently many people (about 0.6% of the total adult population in the UK) with cardiovascular disease are unable to take β-blockers that would prolong their life and improve their cardiovascular symptoms, because of their concomitant respiratory disease. This is because the best β1-selective β-antagonist currently available for clinical use binds to the human β1-adrenoceptor with only 14 fold higher affinity than the human β2-adrenoceptor (Baker, 2005; Br. J Pharmacol; 144, 317-22).

Accordingly there is a need for beta blockers which are selective for just heart disease, i.e. have a high β₁/β₂ selectivity. Classes of phenoxypropanolamine compounds are known which are extended beyond the amine group and are substituted in the phenol ring. One particular class of phenoxypropanolamine compounds comprises a substituted ethylene dioxy substituent para to the phenyl moiety. This class which has never entered into clinical use includes the development compound LK-204545 with an phenyl(alkylurea) substituent to the amine moiety and with 1,778-fold β₁-selectivity:

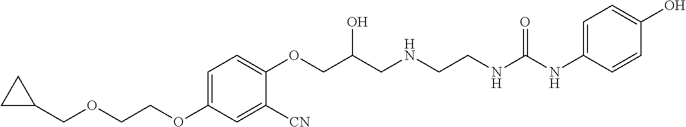 [SMILES: N#Cc1cc(OCCOCC2CC2)ccc1OCC(O)CNCCNC(=O)Nc1ccc(O)cc1]

and D-140S with a phenyl alkyl substituent to the amine moiety and with 4,400-fold β₁-selectivity:

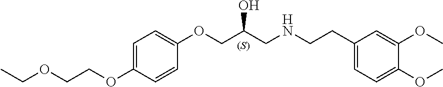

WO2008083054 discloses beta-1 adrenoreceptor selective ligands that find use as imaging agents within nuclear medicine applications. Compounds include an imaging moiety such as a radioactive moiety. The broadly disclosed class of compounds includes compounds having the core 1-phenoxy, 2-hydroxy propan-3-amine with extensive substitution of the phenoxy and amine moieties.

### BRIEF SUMMARY OF THE DISCLOSURE

We have now applied a multidisciplinary approach to beta receptor agonist and antagonist design to provide novel compounds which have significant selectivity for β₁ adrenoceptors and which have potential for clinical use.

### BRIEF DESCRIPTION OF THE DRAWINGS

Embodiments of the invention are further described hereinafter with reference to the accompanying drawings, in which:

Schemes 1,2 and 4 to 7 illustrate synthesis of aryloxypropanolamines of the invention.

### DETAILED DESCRIPTION

In accordance with the present invention there is provided a compound of formula I, and its pharmaceutical acceptable salt or salts and physiologically hydrolysable derivatives in free form or salt form:

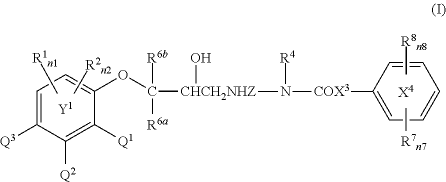

wherein
either Q¹, CR^{6a} and optionally R^{6b} together form a cyclic moiety wherein:
   Q¹ is selected from C₁₋₂ alkylene, C₁₋₂ alkenylene, OC₁ alkylene and OC₁ alkenylene moieties optionally substituted by oxo;
   R^{6a} is a single bond and R^{6b} is H; or
   R^{6a} and R^{6b} together form a double bond; and
   Q² and Q³ are independently selected from H, R¹ and R²;
or Q² and Q³ together form a cyclic moiety in which one of Q² and Q³ is a cyclic moiety selected from OC₁ alkylene and OC₁ alkenylene moieties optionally substituted by oxo or a group R⁵ as hereinbelow defined for R² and the other of Q² and Q³ is a cyclic moiety selected from C₁₋₂ alkylene, C₁₋₂ alkenylene and OC₁ alkylene optionally substituted by oxo;
   R^{6a} and R^{6b} are each H or a cyclic moiety as defined above; and
   Q¹ is selected from H, R¹ and R² and a cyclic moiety as defined above;
and R¹ is independently selected from F, Cl, Br, CN, NH₂, OH, CHO, COOH, CONH₂ and SO₂NH₂;
R² is independently selected from NHR₃, NO₂, CF₃, OR³, COR³, OCOR³, COOR³, CONR³₂, NR³COR³, CONR³₂, SO₂NR³₂, NR³SO₂R³, C₁₋₅alkyl, C₁₋₅alkoxy, C₂₋₅alkenyl, C₂₋₅alkynyl, -Z²-C₃₋₁₀cycloalkyl and -Z²-C₅₋₁₀carbocyclyl wherein Z² is C₁₋₅alkylene, C₂₋₅alkenylene or C₂₋₅alkynylene;
   any of which may comprise one or more carbonyl units or heteroatoms selected from O, S and N and which may be unsubstituted or further substituted by one of more R²¹;
R²¹ is independently selected from C₁₋₅alkyl, C₂₋₅alkenyl, C₂₋₅alkynyl and a group as defined for R¹
   wherein the C₁₋₅alkyl, C₂₋₅alkenyl, C₂₋₅alkynyl groups are optionally substituted by one or more groups independently selected from R¹;
R³ is independently selected from C₁₋₅alkyl; C₁₋₅alkoxy, C₂₋₅alkenyl, C₂₋₅alkynyl, aryl, C₃₋₁₀cycloalkyl, and C₅₋₁₀carbocyclyl;
   any of which may comprise one or more carbonyl units or heteroatoms selected from O, S and N and which may be unsubstituted or further substituted by one or more groups independently selected from R¹;
R⁴ is selected from H and C₁₋₅ alkyl;
n1 and n2 and the sum thereof are independently selected from zero and a whole number integer 1 and 2;
Z is selected from linear C₂₋₃ alkylene;
X³ is NH;
R⁷ is independently selected from F, Cl, Br, CN, NH₂, OH, CHO, COOH, CONH₂ and SO₂NH₂;
R⁸ is independently selected from NHR⁹, NO₂, CF₃, OR⁹, COR⁹, OCOR⁹, COOR⁹, COONR⁹₂, NR⁹COR⁹, CONR⁹₂, SO₂NR⁹₂, NR⁹SO₂R⁹, C₁₋₅alkyl, C₁₋₅alkoxy, C₂₋₅alkenyl, C₂₋₅alkynyl, -Z²-C₃₋₁₀cycloalkyl and -Z²-C₅₋₁₀carbocyclyl wherein Z² is C₁₋₅alkylene, C₂₋₅alkenylene or C₂₋₅alkynylene;
   any of which may comprise one or more carbonyl units or heteroatoms selected from O, S and N and which may be unsubstituted or further substituted by one of more R⁸¹;
R⁸¹ is independently selected from C₁₋₅alkyl, C₂₋₅alkenyl, C₂₋₅-alkynyl and a group as defined for R⁷
   wherein the C₁₋₅alkyl, C₂₋₅alkenyl, C₂₋₅alkynyl groups are optionally substituted by one or more groups independently selected from R⁷;
R⁹ is independently selected from C₁₋₅alkyl, C₁₋₅alkoxy, C₂₋₅alkenyl, C₂₋₅alkynyl, aryl, C₃₋₁₀cycloalkyl, and C₅₋₁₀carbocyclyl;
   any of which may comprise one or more carbonyl units or heteroatoms selected from O, S and N and which may be unsubstituted or further substituted by one of more groups independently selected from R⁷;
n7 and n8 and the sum thereof are independently selected from zero and the whole number integer 1 to 4.

In this embodiment of the above X⁴=Phenyl.

Reference herein to a compound of formula I includes reference to compounds of its subformulae, hereinbelow.

It is to be understood that certain compounds of Formula (I) above may exist in pharmaceutically acceptable salt form or free form. It is to be understood that the present invention encompasses all such salt forms that possess β₁ adrenoceptor activity.

It is to be understood that certain compounds of Formula (I) above may exist in the form of physiologically hydrolysable derivatives in free form or salt form. It is to be understood that the present invention encompasses all such physiologically hydrolysable derivative forms that possess β₁ adrenoceptor activity.

Reference herein to a cyclic moiety is to a moiety which, together with other cyclic moieties, constitutes a cyclic ring.

Compounds of the present invention comprise an optionally substituted bicyclic heteroaromatic ring system comprising benzene ring Y¹ fused to a heterocyclic or heteroaromatic 5 or 6 membered ring, comprising at least one O-heteroatom and optionally including an oxo (═O) moiety, wherein the linking atom for the ring system to the remainder of the compound is a C atom of either ring, and wherein the ring system optionally incorporates the OCR^{6a}R^{6b} moiety.

More preferably in the compounds of the present invention benzene ring Y¹ is fused to the heterocyclic or heteroaromatic ring via heteroatoms formed by the OCR^{6a}R^{6b}, Q¹, Q² and/or Q³ moieties.

Preferably where the ring system is linked to the compound via the heterocyclic ring, it incorporates the OCR^{6a}R^{6b} moiety.

Preferably compounds of formula I wherein the moiety CR^{6a}R^{6b} as hereinbefore defined is CH₂ and Q²Q³Y¹ comprises a heteroaromatic or heterocyclic ring including O heteroatom(s), do not include an imageable entity selected from ¹⁸F, ⁷⁶Br, ¹²⁴⁻⁵I, ¹³¹I, metal chelator or metal chelate complex for an MRI, a ligand for the complexation of a metal for SPECT, a lipid for incorporation into a liposome or the lipid itself.

Preferably a compound of formula I does not include one or more of the following compounds:

| R⁵ | R¹, R² | Q³Q²Y¹Q¹OCR^{6a}R^{6b} | Z | X³ | R⁷, R⁸ |
|---|---|---|---|---|---|
| 2-Ph | — | 7-Che4O—OCH₂ | CH₂CH₂ | NH | 3-Cl |
| — | 5-OCH₂Ph | 2-BzF | CH₂CH₂ | NH | 3-Cl |

wherein R⁴ is H.

Preferably a compound of formula I does not include one or more of the following compounds:

| R⁵ | Q³Q²Y¹Q¹OCR^{6a}R^{6b} | Z | X³ | R⁷, R⁸ |
|---|---|---|---|---|
| 2-OMe | 4-DhBzF—OCH₂ | CH₂CH₂ | NH | —, |
| | | | | 3-F, |
| | | | | 3-Me, |
| | | | | 3-OMe |
| | | | | 2-OMe, 3-OMe |
| — | 5-BzDx-OCH₂, | CH₂CH₂ | NH | — |
| | 4-BzF—OCH₂ | | | |
| | 8-Cha4O—OCH₂ | | | |

wherein R¹, R²=H

In the above table and hereinbelow:
abbreviations have the following meanings:
c. is cyclo; i. is iso; Me or me is methyl; Pr or pr is propyl; Bu or bu is butyl; pent is pentyl; halo is F, Cl, Br or I; Ph is phenyl and Bz is benzyl; o, m and p are ortho, meta and para.; subst. is substituted; o.s. is optionally substituted; - is unsubstituted;
Cha4O=Chroman-4-one Che4O=Chromen-4-one Che2O=chromen-2-one

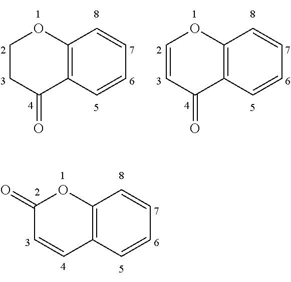

BzF=benzofuran DhBzF=dihydrobenzofuran BzDx=1,4-benzodioxane

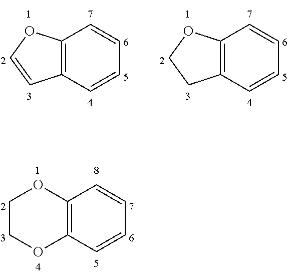 [SMILES: c1ccc2occc2c1 ; c1ccc2c(c1)CCO2 ; c1ccc2c(c1)OCCO2]

In a compound of formula I, the Q³Q²Y¹Q¹ to OCH² linkage may be at any position as indicated above. Preferably in a compound of formula I, Q³Q²Y¹Q¹ is selected from 6-Cha4O, 7-Cha4O, 5-Cha4O, more preferably 7- or 6-; 5-Che4O, 6-Che4O and 8-Che4O, preferably 5- or 6-; 5-Che2O, 6-Che2O, 7-Che2O and 8-Che2O; 5-BzF, 6-BzF and 7-BzF preferably 5- or 6-; 5-DhBzF, 6-DhBzF and 7-DhBzF preferably 5- or 6-; 6-BzDx, 7-BzDx and 8-BzDx preferably 6- or 7-.

In a compound of formula I where OCR^{6a}R^{6b} is a fused cyclic moiety, the linkage to OCH₂CH₂NH etc is typically alpha to —O-heteroatom, eg at the 2-position.

It is to be understood that, insofar as certain of the compounds of Formula I defined above may exist in optically active or racemic forms by virtue of one or more asymmetric carbon atoms, the invention includes in its definition any such optically active or racemic form which possesses adrenoceptor activity. The synthesis of optically active forms may be earned out by standard techniques of organic chemistry well known in the art, for example by synthesis from optically active starting materials or by resolution of a racemic form. Similarly, the above-mentioned activity may be evaluated using the standard laboratory techniques referred to hereinafter.

Examples of suitable methods for separating the enantiomers of a racemic compound include chromatography using a suitable chiral stationary phase: or conversion of a racemic mixture into diastereomeric derivatives, separation of the mixture of diastereomeric derivatives into two single diastereomers, and regeneration of a separate single enantiomer from each separate single diastereomer.

Examples of suitable methods for separating a mixture of diastereomers include fractional crystallisation, normal-phase chromatography, or reverse-phase chromatography.

It is to be understood that certain compounds of Formula I defined above may exhibit the phenomenon of tautomerism. In particular, tautomerism may affect any heterocyclic groups that bear 1 or 2 oxo substituents. It is to be understood that the present invention includes in its definition any such tautomeric form, or a mixture thereof, which possesses β₁ adrenoceptor activity and is not to be limited merely to any one tautomeric form utilised within the formulae drawings or named in the Examples.

It is to be understood that certain compounds of Formula (I) above may exist in unsolvated forms as well as solvated forms, such as, for example, hydrated forms. It is to be understood that the present invention encompasses all such solvated forms that possess β₁ adrenoceptor activity.

It is also to be understood that certain compounds of the Formula (I) may exhibit polymorphism, and that the present invention encompasses ail such forms which possess β₁ adrenoceptor activity.

In preferred embodiments there is provided a compound of subformula Ia, Ib or Ic and their pharmaceutically acceptable salt or salts and physiologically hydrolysable derivatives:

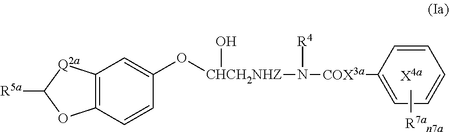

wherein
Q^{2a} is selected from COCH₂, COCH, CHCH, CH₂, CH and OCH₂;
R^{5a} is selected from Ph, oxo, alkyl, alkoxyalkyl CO₂NH₂ and CO₂alkyl;
X^{3a} is NH;
R^{7a} is 3-Cl or 4-OH; and
n7a is 1;

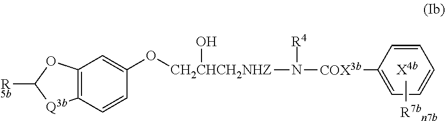

wherein
Q^{3b} is selected from COCH₂ and OCH₂;
R^{5b} is selected from Ph and CH₂OC₂H₅;
X^{3b} is NH;
R^{7b} is 3-Cl or 4-OH; and
n7b is 1; and

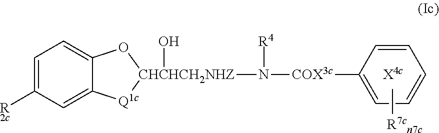

wherein
Q^{1c} is selected from CH₂CH₂ CH₂, CH₂CH, CH, CHCH, OCH₂, COCH₂ and COCH;
R^{2c} is selected from H and cycloalkoxyalkoxyl;
X^{3c} is NH;
R7c is 3-Cl or 4-OH; and
n7c is 1;
wherein other integers are as hereinbefore defined for compounds of formula (I).

Most preferably the Q²Q³Y¹Q¹OCR^{6a}R^{6b} moiety is selected from 4-chromanon-7-yloxymethylene, 4-chromenon-6-yloxymethylene, 2-chromenon-6-yl, benzofuran-5-yloxymethylene, benzofuran-2-yl, dihydrobenzofuran-5-yloxymethylene, dihydrobenzofuran-2-yl, 1,4-benzodioxan-2-yl and 1,4-benzodioxan-6-yloxymethylene.

In a further embodiment of the invention R², Q²R⁵ or Q³R⁵ comprises a group or moiety OZ¹OR⁴ in which
Z¹ is C₁-C₄ branched or linear alkyl or alkenyl;
R⁴ is selected from unsubstituted and substituted C₁-C₄ linear or branched alkyl, C₁₋₅ alkenyl, C₅-C₁₀ heteroaryl or aryl, C₃-C₈ cycloalkyl or heterocyclyl which may be part unsaturated, and combinations thereof; wherein substituents include any of R¹ and R² as hereinbefore defined.

Table 1 below gives representative compounds of formula I which are illustrative only and not intended to be exclusive:

TABLE 1

| Ex. | R⁵ | R¹, R² | Q³Q²Y¹Q¹OCR^{6a,6b} | Z | X³ | R⁷, R⁸ |
|---|---|---|---|---|---|---|
| 4a | 2-Ph | — | 7-Cha4O—OCH₂ | (CH₂)₂ | NH | 3-Cl |
| 4b | 2-Ph | — | 6-Che4O—OCH₂ | (CH₂)₂ | NH | 3-Cl |
| 4c | — | — | 6-Che2O—OCH₂ | (CH₂)₂ | NH | 3-Cl |
| 4d | — | — | 6-Che2O-(4-Me)—OCH₂ | (CH₂)₂ | NH | 3-Cl |
| 24a | 2-CO₂C₂H₅ | — | 5-BzF—OCH₂ | (CH₂)₂ | NH | 3-Cl |
| 24b | 2-CO₂NH₂ | — | 5-BzF—OCH₂ | (CH₂)₂ | NH | 3-Cl |
| 46a | — | — | 2-BzDx | (CH₂)₂ | NH | 3-Cl |
| 46b | — | — | 2-BzDx | (CH₂)₂ | NH | 4-OH |
| 63a | — | 5-O(CH₂)₂Oc • pent | 2-DhBzF | (CH₂)₂ | NH | 4-OH |
| 63b | — | 5-O(CH₂)₂Oc • pent | 2-BzF | (CH₂)₂ | NH | 4-OH |
| 63c | — | 6-CH₃ | 2-BzF | (CH₂)₂ | NH | 4-OH |
| 63d | — | 5-OCH₃ | 2-BzF | (CH₂)₂ | NH | 4-OH |
| 76 | 2-CH₂OC₂H₅ | — | 7-BzDx-OCH₂ | (CH₂)₂ | NH | 4-OH |
| 610a | 2-CH₂OC₂H₅ | — | 5-BzF—OCH₂ | (CH₂)₂ | NH | 4-OH |
| 610b | 2-CH₂OC₂H₅ | — | 5-DhBzF—OCH₂ | (CH₂)₂ | NH | 4-OH |
| 610c | 2-CH₃ | — | 5-BzF—OCH₂ | (CH₂)₂ | NH | 4-OH |

wherein R⁴ is H and X⁴ is phenyl.

A compound as hereinbefore defined may be in free form, i.e. normally as a base, or in any suitable salt or ester form. Free forms of the compound may be converted into salt or ester form and vice versa, in conventional manner. Suitable salts include hydrochloride, dihydrochloride, hydroformate, amide, succinate, half succinate, maleate, acetate, trifluoroacetate, fumarate, phthalate, tetraphthalate, benzoate, sulfonate, sulphate, phosphate, oxalate, malonate, hydrogen malonate, ascorbate, glycolate, lactate, malate, tartarate, citrate, aspartate or glutamate and variants thereof. Suitable acids for add addition salt formation include the corresponding acids, i.e. hydrochloric, formic, amino acid, succinic, maleic, acetic, trifluoroacetic, fumaric, phthalic, tetraphthalic, benzoic, sulfonic, sulphuric, phosphoric, oxalic, malonic, ascorbic, glycolic, lactic, malic, tartaric, citric, aspartic or glutamic acids and the like.

Suitable esters include those obtained with the above acids, with hydroxides such as sodium, potassium, calcium or the like, or with alcohols.

The compounds of formula I and subformulae are optically active and may be prepared as one or both enantiomeric or tautomeric forms, or stereo or geometric isomeric forms, where relevant. Such forms may be identified and prepared or isolated by methods known in the art. Reference herein to compounds of formula I also encompasses reference to crystalline forms, polymorphs, hydrous and anhydrous forms and prodrugs thereof.

In a further aspect of the invention there is provided a process for the preparation of a compound of formula I or subformulae as hereinbefore defined comprising reacting a compound of formula LII or LV:

Q³Q²Y¹Q¹OCR^{6a}R^{6b}oxirane LII

Q³Q²Y¹Q¹OCR^{6a}R^{6b}CORCH₂R^{L} where R=a bond and R^{L}=Br LV

with a compound of formula RII;

H₂NZNHCOX³X⁴. RII

A compound of formula LII is conveniently prepared by methods described in GB 0911657 from the corresponding LIII or is commercially available:

Q³Q²Y¹Q¹R where R=OH. LIII

LIII is conveniently obtained by interchange from a commercially available analogue or is commercially available.

A compound of formula LIII where Q³Q²Y¹Q¹OCR^{6a}R^{6b} is 5-“DhBzF”OCH₂ or 5-“DhBzF”OCH₂ is conveniently obtained from the corresponding LIII above where R=OH, in turn from LIII where R=OCH₂Ph, in turn obtained from reaction of LIV with acetate:

2-CHO, 3-OH PhR where R=OCH₂Ph. LIV

LIV is conveniently obtained from the corresponding LIV having protected OH (3-OCH₂Ph), in turn from diol LIV where R=OH, commercially available.

A compound of formula LIII where Q³Q²Y¹Q¹OCR^{6a}R^{6b} is 2-BzD is conveniently obtained from the corresponding LV:

Q³Q²Y¹Q¹OCR^{6a}R^{6b}CORCH₂R^{L} where R=H and R^{L}=Br. LV

A compound of formula LV is conveniently obtained from the corresponding LV where R=a bond and R^{L}=Br, in turn from, or directly from, LV where R=a bond and R^{L}=H.

A compound of formula LV where R=a bond and R^{L}=H is commercially available (where Q³Q²Y¹Q¹OCR^{6a}R^{6b} is 2-BzD) or where Q³Q²Y¹Q¹OCR^{6a}R^{6b} is 2-BzF is obtained by cyclisation of a protected compound of formula LVI:

(OH)(OP)Y¹R where P=TBS and R=OH, in turn from the corresponding unprotected LVI, commercially available. LVI

A compound of formula LIII where Q³Q²Y¹Q¹OCR^{6a}R^{6b} is 6-BzD is conveniently obtained from the corresponding LIII where R=OH, in turn the corresponding LIII where R=CHO, in turn obtained by cyclisation of a compound of formula LVI:

(OH)₂Y¹R where (OH)₂ are at 3,4-positions and R=CHO, commercially available. LVI

A compound of formula RII is commercially available or is obtained from reaction of the corresponding RV and CII:

COX³X⁴ where X³ is ═N RV

H₂NZNH-Boc CII

Suitably a process is as hereinbefore defined or as hereinbelow illustrated in the drawings.

In a further aspect of the invention there is provided a novel intermediate as hereinbefore defined. Preferably a novel intermediate is of formula LII, LIII, LIV, LV, LVI, RII and RV as hereinbefore defined. Novel intermediates include 1, 2, 3, 21 22, 23, 42, 43, 44, 45a, 45b, 52, 53, 54, 55, 57, 58, 59, 60, 602, 603, 604, 605, 606, 607a,b,c, 608a,b,c, 609, 72, 73 and 74 as hereinbelow defined.

In a further aspect of the invention there is provided a process as hereinbefore defined for the preparation of a novel intermediate as hereinbefore defined or as hereinbelow illustrated in the drawings.

### Therapeutic Use

In a further aspect of the invention there is provided a compound of formula I or subformulae as hereinbefore defined for use as a medicament.

In a further aspect of the invention there is provided the use of a compound of formula I or subformulae as hereinbefore defined in the prevention or treatment of a condition selected from ischaemic heart disease (also known as myocardial infarction or angina), hypertension and heart failure, restenosis and cardiomyopathy, more preferably with concomitant respiratory disease, in particular asthma or COPD.

In a further aspect of the invention there is provided the use of a compound of formula I or subformulae as hereinbefore defined in the manufacture of a medicament for prevention or treatment of a condition selected from ischaemic heart disease (also known as myocardial infarction or angina), hypertension and heart failure, restenosis and cardiomyopathy, more preferably with concomitant respiratory disease, in particular asthma or COPD.

In a further aspect of the invention there is provided a compound of formula I or subformulae as hereinbefore defined for the prevention or treatment of a condition selected from ischaemic heart disease (also known as myocardial infarction or angina), hypertension and heart failure, restenosis and cardiomyopathy, more preferably with concomitant respiratory disease, in particular asthma or COPD.

In a further aspect of the invention there is provided a method of treating a condition selected from ischaemic heart disease (also known as myocardial infarction or angina), hypertension and heart failure, restenosis and cardiomyopathy, more preferably with concomitant respiratory disease, in particular asthma or COPD, said method comprising administering to a subject in need thereof, a compound of formula I or subformulae or pharmaceutically acceptable salt thereof as hereinbefore defined in an amount sufficient to treat the condition.

In a further aspect of the invention there is provided a method of preventing a condition selected from ischaemic heart disease (also known as myocardial infarction or angina), hypertension and heart failure, restenosis and cardiomyopathy, more preferably with concomitant respiratory disease, in particular asthma or COPD, said method comprising administering to a subject in need thereof, a compound of formula I or subformulae or pharmaceutically acceptable salt thereof as hereinbefore defined in an amount sufficient to treat the condition.

The use of a compound of the invention in the manufacture of a medicament as hereinbefore defined includes the use of the compound directly, or in any stage of the manufacture of such a medicament, or in vitro in a screening programme to identify further agents for the prevention or treatment of the hereinbefore defined diseases or conditions.

A further aspect of the invention relates to the use of a compound of formula I or a pharmaceutically acceptable salt or solvate or physiologically hydrolysable, solubilising or immobilising derivative thereof, in an assay for identifying candidate compounds capable of treating one or more disorders or diseases as hereinbefore defined.

### Pharmaceutical Compositions

In a further aspect of the invention there is provided a composition comprising a therapeutically effective amount of a compound of formula I or subformulae or its pharmaceutically acceptable salt or physiologically hydrolysable derivative as hereinbefore defined in association with one or more pharmaceutical carriers, excipients or diluents. Suitable carriers, excipients or diluents may be selected having regard to the intended mode of administration and standard practice. The pharmaceutical compositions may be for human or animal usage in human and veterinary medicine, preferably for treatment of a condition, disease or disorder as hereinbefore defined

Examples of suitable carriers include lactose, starch, glucose, methyl cellulose, magnesium stearate, mannitol, sorbitol and the like.

A composition or compound of the invention is suitably for any desired mode of administration including oral, rectal, vaginal, parenteral, intramuscular, intraperitoneal, intraarterial, intrathecal, intrabronchial, subcutaneous, intradermal, intravenous, nasal, buccal or sublingual and the like. An indicated daily dosage is from about 1 mg to about 500 mg and compositions for oral administration generally contain from about 0.25 mg to about 250 mg of the compound together with solid or liquid carriers and diluents. A therapeutically effective amount is any amount from 0.1% to 99.9% w/w.

A composition for oral administration is suitably formulated as a compressed tablet, tablet, capsule, gel capsule, powder, solution, dispersion, suspension or the like. Such forms may be produced according to known methods and may include any suitable binder, lubricant, suspending agent, coating agent or solubilising agent or combinations thereof.

A composition for administration by means of injection is suitably formulated as a sterile solution or emulsion from a suitable solution or powder. Alternatively a composition may be in the form of suppositories, pessaries, suspensions, emulsions, lotions, creams, ointments, skin patches, gels, solgels, sprays, solutions or dusting powders.

A composition may include one or more additional active ingredients or may be administered together with compositions comprising other active ingredients for the same or different condition. An additional active ingredient is suitably selected from a diuretic, calcium channel antagonist, angiotensin converting enzyme (ACE) inhibitor, angiotensin receptor antagonist and the like.

The compounds of the invention may be administered in the form of a pro-drug, that is a compound that is physiologically hydrolysable in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I or subformulae as hereinbefore defined and in vivo cleavable amide derivatives that may be formed at a carboxy group or an amino group in a compound of the Formula I or subformulae as hereinbefore defined.

Accordingly, the present invention includes those compounds of the Formula I or subformulae as defined hereinbefore when made available by organic synthesis and when made available within the human or animal body by way of cleavage of a pro-drug thereof. Accordingly, the present invention includes those compounds of the Formula I or subformulae as hereinbefore defined that are produced by organic synthetic means and also such compounds that are produced in the human or animal body by way of metabolism of a precursor compound, that is a compound of the Formula I or subformulae as hereinbefore defined 1may be a synthetically-produced compound or a metabolically-produced compound.

A suitable pharmaceutically-acceptable pro-drug of a compound of the Formula I or subformulae as hereinbefore defined is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 ‘Design and Application of Pro-drugs’, by H, Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al. Chem. Pharm. Bull., 32, 692 (1984);
g) T. Higuchi and V. Stella, ‘Pro-Drugs as Novel Delivery Systems’, A.C.S. Symposium Series, Volume 14; and
h) E. Roche (editor), ‘Bioreversible Carriers in Drug Design’, Pergamon Press, 1987.

A suitable pharmaceutically-acceptable pro-drug of a compound of the Formula I or subformulae as hereinbefore defined that possesses a carboxy group is, for example, an in vivo cleavable ester thereof. An in vivo cleavable ester of a compound of the Formula I or subformulae as hereinbefore defined containing a carboxy group is, for example, a pharmaceutically-acceptable ester which is cleaved in the human or animal body to produce the parent acid. Suitable pharmaceutically-acceptable esters for carboxy include C₁₋₆alkyl esters such as methyl, ethyl and tert-butyl, C₁₋₆alkoxymethyl esters such as methoxymethyl esters, C₁₋₆alkanoyloxymethyl esters such as pivatoyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-yl methyl esters and C₁₋₆alkoxycarbonyloxy-C₁₋₆alkyl esters such as methoxycarbonyloxymethyl and 1-methoxycarbonyloxyethyl esters.

A suitable pharmaceutically-acceptable pro-drug of a compound of the Formula I or subformulae as hereinbefore defined that possesses a hydroxy group is, for example, an in vivo cleavable ester or ether thereof.

An in vivo cleavable ester or ether of a compound of the Formula I or subformulae as hereinbefore defined containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include C₁₋₁₀alkanoyl groups such as acetyl, benzoyl, phenylacetyl and substituted benzoyl and phenylacetyl groups, C₁₋₁₀-alkoxycarbonyl groups such as ethoxycarbonyl, N,N-[di-C₁₋₄alkyl]carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-C₁₋₄alkylpiperazin-1-ylmethyl. Suitable pharmaceutically-acceptable ether forming groups for a hydroxy group include alpha-acyloxyalkyl groups such as acetoxymethyl and pivaloyloxymethyl groups.

A suitable pharmaceutically-acceptable pro-drug of a compound of the Formula (I) that possesses a carboxy group is, for example, an in vivo cleavable amide thereof, for example an amide formed with an amine such as ammonia, a C₁₋₄alkylamine such as methylamine, a di-C₁₋₄alkylamine such as dimethylamine, N-ethyl-N-methylamine or diethylamine, a C₁₋₄alkoxy-C₂₋₄alkylamine such as 2-methoxyethylamine, a phenyl-C₁₋₄alkylamine such as benzylamine and amino acids such as glycine or an ester thereof.

A suitable pharmaceutically-acceptable pro-drug of a compound of the Formula (I) or subformulae as hereinbefore defined that possesses an amino group is, for example, an in vivo cleavable amide derivative thereof. Suitable pharmaceutically-acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄)alkylpiperazin-1-ylmethyl.

In a further aspect of the invention there is provided the use of a compound of formula I or subformulae or a composition as hereinbefore defined in the prevention or treatment of a condition selected from ischaemic heart disease (also known as myocardial infarction or angina), hypertension and heart failure, in a particular advantage a compound or composition of the invention may be administered to a subject with, or used in the prevention or treatment of a subject suffering from one of the above conditions and from respiratory disease, in particular from asthma or COPD. In a further advantage a compound or composition of the invention may be administered to a subject with, or used in the prevention or treatment of a subject suffering from one of the above conditions and intolerant to a side effect associated with known beta blockers. In a further advantage a compound or composition of the invention has good oral bioavailability.

We have found that the compounds and compositions of the invention block beta-1 mediated responses but have substantially no affect on beta-2 mediated responses in a conscious animal. The beta-1 mediated responses include tachycardia, reflex heart rate response etc and the like, and are implicated in the above conditions. The beta-2 mediated responses include peripheral vascular conductance, hypotension and the like and are implicated in respiratory conditions.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

### Experimental—Abbreviations

1°, primary; 4°, quaternary; Ar, aromatic ring; Boc, tert-butylcarbonate; Boc₂O, di-tert-butyl dicarboxylate; br, broad; brine, saturated sodium chloride solution; C, carbon; cAMP, cyclic adenosine monophosphate; CDCl₃, deuterated chloroform; COMFA, comparative molecular field analysis; COSY, correlation spectroscopy; d, doublet; D₂O, deuterated water; DCC, dicyclohexylcarbodiimide: DCM, dichloromethane; dd, doublet of doublets; DEAD, diethyl azodicarboxylate; def, deformation; DEPT, distortionless enhanced polarisation transfer; DMF, N,N-dimethylformamide; DMSO, dimethyl sulphoxide; DMSO-d₆, deuterated dimethyl sulphoxide; DPPA, Diphenylphosphoryl azide; dt, doublet of triplets; EDC, 1-ethyl-3-(3-dimethylaminopropyl) carbodiimide hydrochloride; eq, molar equivalents; ES, electrospray; Et₂O, diethyl ether; EtOAc, ethyl acetate; EtOH, ethanol; FA, formic acid; FT-IR, fourier transform—Infrared; H₂, hydrogen gas; HCl hydrochloric acid; HMBC, heteronuclear multiple bond correlation; HPLC, high performance liquid chromatography; HSQC, heteronuclear single quantum correlation; J, Coupling constant; J_{CF}, Carbon-Fluorine coupling constant; K₂CO₃, Potassium carbonate; KHSO₄, potassium hydrogen sulfonate; KMnO₄, potassium permanganate; lit, literature; m, multiplet; MeCN, acetonitrile; MeOH, methanol; MgSO₄, anhydrous magnesium sulphate; Mp, melting point; MS, mass spectrometry; MW, microwave; m/z, observed ion; NaH, sodium hydride; NaHCO₃, Sodium Hydrogen Carbonate; NaOH, sodium hydroxide, NH₃, Aqueous ammonia solution (35%); NMR, nuclear magnetic resonance spectroscopy; Pd, palladium; PDE, phosphodiesterase; phth, phthalimide; PLC, preparative layer chromatography; PMA, phosphomolybdic acid; ppm, parts per million; PPTS, pyridinium para-tolueunesulphonate; ^{c}Pe, cyclopentyl; ^{c}Pr, cyclopropyl; p-TsCl, para-toluene sulfonylchloride; q, quadruplet: Rₜ, retention time; s, singlet; str, stretch; t, triplet; TEA, triethylamine; TFA, trifluoroacetic acid; THF, tetrahydrofuran; THP, tetrahydropyran; TMS, tetramethylsilane; TOF, time of flight.

### General Chemistry

Chemicals and solvents were purchased from standard suppliers and used without further purification. Merck Kieseigel 60, 230-400 mesh, for flash column chromatography was supplied by Merck KgaA (Darmstadt, Germany) and deuterated solvents were purchased from Goss International Limited (England) and Sigma-Aldrich Company Ltd (England).

Unless otherwise stated, reactions were carried out at ambient temperature. Reactions were monitored by thin layer chromatography on commercially available precoated aluminium backed plates (Merck Kieseigel 60 F₂₆₄). Visualisation was by examination under UV light (254 and 366 nm). General staining carried out with Ninhydrin, KMnO₄ or PMA. Or LC MS (see method below)

All organic extracts after aqueous work-up procedures were dried over MgSO₄ or Na₂SO4 before gravity filtering and evaporation to dryness. Organic solvents were evaporated under reduced pressure at ≦40° C. (water bath temperature). Purification using preparative layer chromatography was carried out using Fluka silica gel 60 PF₂₅₄ containing gypsum (200 mm×200 mm×1 mm). Flash chromatography was performed using Merck Kieseigel 60 (0.040-0.063 mm). Or using Flashmaster or Isolera 4

Melting points were recorded on a Reichert 7905 apparatus or Perkin Elmer Pyris 1 differential scanning calorimeter and were uncorrected. FT-IR spectra were recorded as thin films or KBr discs in the range of 4000-500 cm⁻¹ using and Avatar 360 Nicolet FT-IR spectrophotometer. Optical rotation was measured on a Bellingham-Stanley ADP220 polarimeter.

Melting points were recorded on an Electrothermal melting point apparatus or Mettier Toledo

Melting Point System MP50 and were uncorrected.

Mass spectra (TOP ES +/−) were recorded on a Waters 2795 separation module/micromass LCT platform.

¹H NMR spectra were recorded on a Bruker-AV 400 at 400.13 MHz, ¹³C NMR spectra were recorded at 101.62 MHz. Chemical shifts (δ) are recorded in ppm with reference to the chemical shift of the deuterated solvent/an internal TMS standard. Coupling constants (J) are recorded in Hz and the significant multiplicites described by singlet (s), doublet (d), triplet (t), quadruplet (q), broad (br), multiplet (m), doublet of doublets (dd), doublet of triplets (dt). Spectra were assigned using appropriate COSY, DEPT, HSQC and HMBC sequences. Unless otherwise stated all spectra were recorded in CDCl₃.

Analytical HPLC were performed on a Shimadzu UFLCXR system coupled to an Applied Biosystems API2000. Two columns thermostated at 40° C. were used.

Colunm one: Gemini-NX 3u-110A, 50×2 mm

Column two: Luna 3u (PFP2) 110A, 50×2 mm.

Flow rate 0.5 ml/min, UV detection at 220 and 254 nm.

Gradient: Pre-equilibration run for one min at 10% B, 10 to 98% solvent B in 2 min, 98% for 2 min, 98 to 10% B in 0.5 min then 10% for one min.

Solvent A: 0.1% Formic Acid in water; solvent B: 0.1% Formic Acid in MeCN.

### EXAMPLE 1

### Synthesis of Chromanyl and Chromenyl Analogues of Aryloxypropanolamines

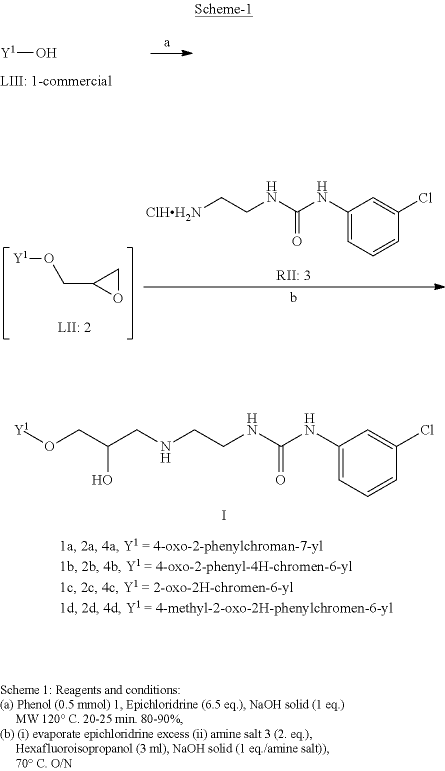

Using the general synthesis disclosed in UK Patent Application No 0911857.5 and shown in Scheme 1, the following compounds were prepared:

Phenol 1a-1d (0.5 mmol) was dissolved in epichloridrine (6.5 eq,, 3.2 mmol, 250 ul) and NaOH solid (1 eq., 0.5 mmol, 20 mg) added. The reaction was heated by MW at 120° C. for 25 min. The reaction was analysed by LC MS and excess epichloridrine was evaporated using the Genevac, full vacuum and NO heat.

After evaporation, HFIP (3 ml), amine salt 3 (2 eq., 500 mg) and NaOH solid (2 eq., 40 mg) were added and the suspension stirred at 70° C. O/N.

The crude HFIP suspension was wet loaded on isolute cartridge (silica, 10 g) and dried under vacuum before purification by flash master using a gradient DCM/1M NH3 in MeOH. Purification was followed by TLC and LCMS.
4a: 1-(3-chlorophenyl)-3-(2-(2-hydroxy-3-(4-oxo-2-phenylchroman-7-yloxy)propylamino)ethyl)urea
4b: 1-(3-chlorophenyl)-3-(2-(2-hydroxy-3-(4-oxo-2-phenyl-4H-chromen-6-yloxy)propylamino)ethyl)urea
4c: 1-(3-chlorophenyl)-3-(2-(2-hydroxy-3-(2-oxo-2H-chromen-6-yloxy)propylamino)ethyl)urea
4d: 1-(3-chlorophenyl)-3-(2-(2-hydroxy-3-(4-methyl-2-oxo-2H-chromen-6-yloxy)propylamino)ethyl)urea

### EXAMPLE 2

### Synthesis of 2-Substituted Benzofuran Analogues of Aryloxypropanolamines

a) Ethyl-5-hydroxybenzofuran-2-carboxylate 21

Starting from ethyl-5-(benzyloxy)benzofuran-2-carboxylate (obtained by part hydrogenation of 604, synthesis below)

b) Ethyl-5-(oxiran-2-ylmethoxy)benzofuran-2-carboxylate 22 was prepared by analogy with Example 1 using the materials in Scheme 2.

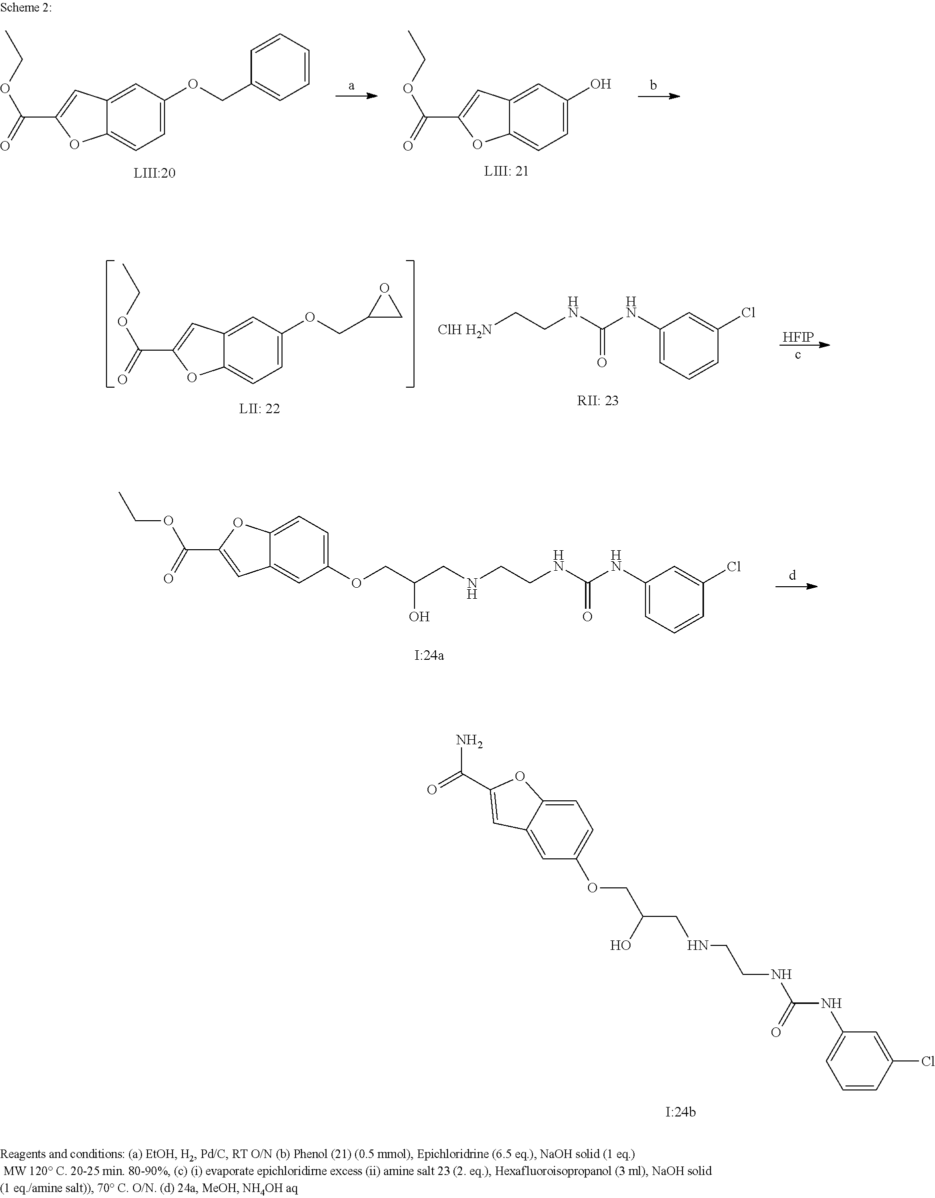

c) Ethyl-5-(3-(2-(3-(3-Chlorophenyl)ureido)ethylamino)-2-hydropropoxy)benzofuran-2-carboxylate, 24a

The epoxide 22 (150 mg, 0.5 mmol) is dissolved in HIPF (4 ml), amine salt 23 1-(2-aminoethyl)-3-(3-chlorophenyl)urea hydrochloride (1.2 eq., 0.6 mmol, 110 mg) and NaOH (solid, 1.2 eq., 0.6 mmol, 24 mg) are added and heat at 70° C. ON. The suspension is transferred directly on a Flashmaster silica cartridge 10 g/70 ml and chromatographied using a gradient DCM/2M NH3 MeOH to get an orange oil 24a, m=29 mg

d) 5-(3-(2-(3-(3-chlorophenyl)ureido)ethylamino)-2-hydropropoxy)benzofuran-2-carboxamide, 24b

24a (25 mg) is dissolved in MeOH (2 ml) and CH₃NH (40% sol in water, 1 ml) is added. The vessel is tightly closed and solution stirred O/N at RT. Evaporate to dryness to get an orange film. The film is re-dissolved in MeOH (1-2 drops) and TBME is added to form a white suspension. The suspension is left at RT O/N to rest, and is centrifuge to separate the solid. A Final purification is carried cut by re-crystallisation in water (with one drop NaOH, 2M) to remove traces of carboxylic acid. Centrifuge the solid and dry under vacuum to obtain 24b.

### EXAMPLE 4

### Synthesis of 2,3-dihydrobenzo[b][1,4]dioxin-2-yl Analogues of aryloxypropanolamines (Scheme 4)

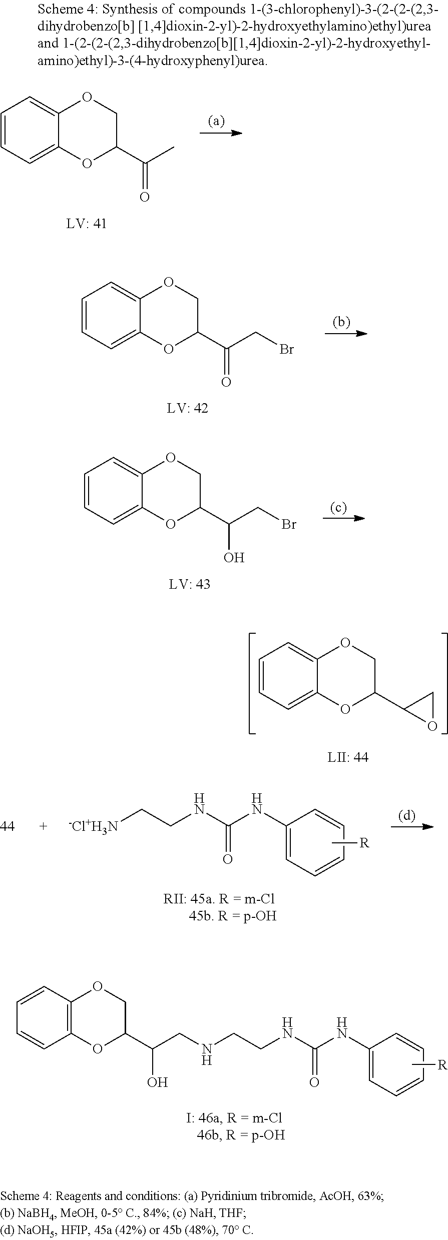

a) 2-bromo-1-(2,3-dihydrobenzo[b][1,4]dioxin-2-yl)ethanone 42:

Pyridinium tribromide (1.46 g, 4.566 mmol) was suspended in AcOH (15 mL), and added over 20 minutes to a stirred solution of 2-acetylbenzodioxane 41 (678 mg, 3.805 mmol) in acetic acid (55 mL) at room temperature during 80 minutes. Water (100 mL) was added and the mixture was extracted with diethyl ether (2×170 mL), the combined organic layers extract were washed with a saturated solution of sodium hydrogenocarbonate (5×40 mL), water (20 mL), and dried over Na₂SO₄. After concentration of the filtrate, purification was achieved via FCC (eluent: Petroleum Ether-DCM 50:50) to give a clear oil, which after trituration in Petroleum Ether and filtration afforded 42 as a white solid (612 mg, 63% yield).

b) 2-bromo-1-(2,3-dihydrobenzo[b][1,4]dioxin-2-yl)ethanol 43:

To a solution of 42 (405 mg, 1.576 mmol) in MeOH (10 mL) at 0-5° C. was added NaBH₄ (30 mg, 0.788 mmol). After 10 minutes, the mixture was reduced to a small volume and directly purified by FCC (eluent: Petroleum Ether-DCM 30:70) to afford 43 (clear oil, 344 mg, 84% yield) as a mixture of diastereoisomers (3:1).

c) 2-(oxiran-2-yl)-2,3-dihydrobenzo[b][1,4]dioxine 44:

Under nitrogen, a solution of 43 in dry THF (10 mL) was added over 5 min to a suspension of washed NaH (3 times with Petroleum Ether) in dry THF (8 mL), producing a hydrogen degassing. The white suspension was stirred overnight, filtered, evaporated and dried under high-vacuum to afford 257 mg of crude 44, which was used in the next step without further purification.

d) 1-(3-chlorophenyl)-3-(2-(2-(2,3-dihydrobenzo[b][1,4]dioxin-2-yl)-2-hydroxyethylamino)ethyl)urea 46a:

A solution of crude 44 (118 mg) was dissolved in HFIP (5 mL). Then the 1-(2-aminoethyl)-3-(3-chlorophenyl)urea hydrochloride 45a (331 mg, 1.324 mmol), and NaOH₄ (53 mg, 1.324 mmol) was added and the mixture stirred at 70° C. during 1 day. The whole suspension was slowly loaded at the top of a silica column, dried, and purified by FCC (eluent DCM-1M NH3 in MeOH 90:10) to afford 46a (white solid, 110 mg, over 2 steps) as a mixture of diastereoisomers (3:1).

1-(2-(2-(2,3-dihydrobenzo[b][1,4]dioxin-2-yl)-2-hydroxyethylamino)ethyl)-3-(4-hydroxyphenyl)urea 46b:

Synthesized according to the procedure used for 46a using the 1-(2-aminoethyl)-3-(4-hydroxyphenyl)urea hydrochloride 45b. 46b (white solid, 119 mg, over 2 steps) was obtained as a mixture of diastereoisomers (3:1).

### EXAMPLE 5

### Synthesis of 2,3-dihydrobenzofuran-2-yl and benzofuran-2-yl Analogues of aryloxypropanolamines (Scheme 5)

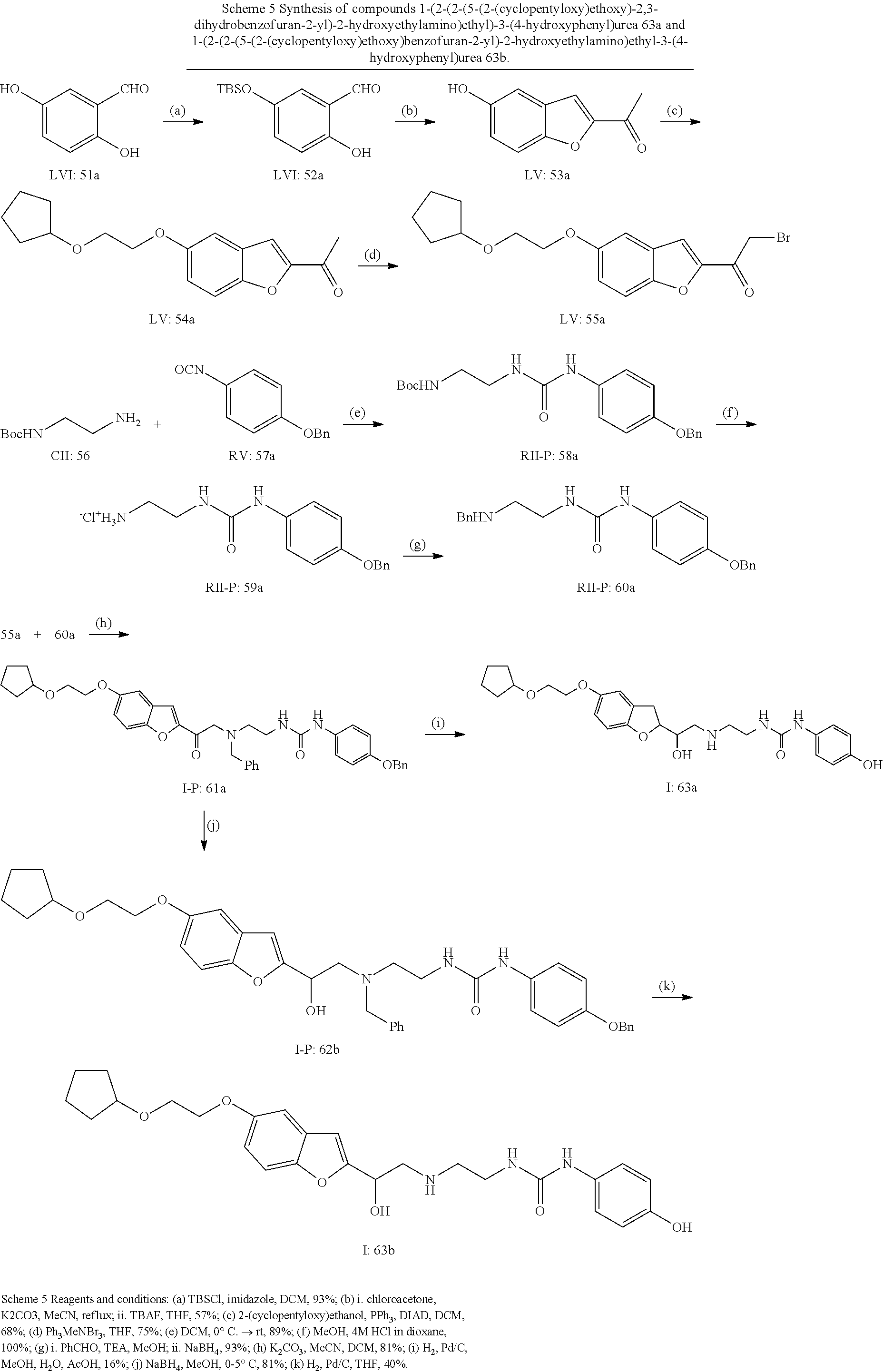

a) 5-(tert-butyldimethylsilyloxy)-2-hydroxybenzaldehyde 52a:

Imidazole (15.53 g, 0.228 mol) and 2,5-dihydroxybenzaldehyde (15.0 g, 0.109 mol) were dissolved in DCM (250 mL) to give a brown solution. TBSCl (18.0 g, 0.199 mol) was added in portions and the mixture was stirred at room temperature overnight. After removal of all volatiles under reduced pressure, the crude residue was dispersed between petroleum ether (100 mL) and wafer (100 mL). The aqueous layer was extracted with petroleum ether (3×100 mL), before concentration of the combined organic layers to give 27.78 g of dark green oil. This was further purified by FCC (eluent TBME/PE 2:98), to afford 25.54 g (93% yield) of 52a as a yellow oil.

b) 1-(5-hydroxybenzofuran-2-yl)ethanone 53a:

To a solution of 52a (7.94 g, 0.031 mol) in MeCN (350 mL) was added K₂CO₃ (13.04 g, 0.094 mol). The chloroacetone (3.01 mL, 0.038 mol) was added dropwise, and then the suspension was stirred 5 hours under reflux, allowed to cool down, filtered, and the solvent was evaporated under reduced pressure to afford a dark brown oil. This one was further filtered through a silica plug with DCM to afford, after evaporation of the solvent, 7.5 g of crude 1-(5-(tert-butyldimethylsilyloxy)benzofuran-2-yl)ethanone as orange oil. This last one was then dissolved in THF (300 mL), TBAF (10.85 g, 0.041 mol) was added dropwise, and the resulting solution was stirred over-night. The mixture was then extended with 250 mL of water and the THF removed under reduced pressure. The aqueous layer was extracted with 2×200 mL of DCM, saturated with solid NaCl and extracted once more with 200 mL of DCM. The combined organic layers were rinsed with 2×50 mL of brine, dried over Na₂SO₄, and filtered. The solvent was evaporated and the residue purified by FCC (eluent DCM-MeOH 95:5) to obtain 3.67 g of a brown solid containing 53a in a mix with 15% of TBAF impurities. A dissolution of this solid in 1M NaOH (150 mL), followed by reacidification (pH=2) with concentrate HCl allowed the precipitation of 53a, filtered and obtained as a pale brown solid (3.12 g, 57% yield).

c) 1-(5-(2-(cyclopentyloxy)ethoxy)benzofuran-2-yl)ethanone 54a:

To a stirred suspension of 53a (850 mg, 4.825 mmol), 2-(cyclopentyloxy)ethanol (817 mg, 6.275 mmol) and triphenylphosphine (2.214 g, 8.444 mmol) in DCM (250 mL) was added dropwise a solution of DIAD (1.707 g, 8.444 mmol) in DCM (40 mL), leading to the dissolution of the suspension, which was further stirred overnight, washed with 2×50 mL of 1M NaOH and 2×20 mL of brine. The organic layer was then evaporated until a weak volume. On addition of a Petroleum Ether-Et₂O solution (300 mL, 8:2) a precipitate of triphenylphosphine oxide began to form. The flask was left in the freezer for 1 hour before filtration of the precipitate and washing with Petroleum Ether and Et₂O. After concentration of the filtrate, purification was achieved via FCC (eluent: Petroleum Ether-EtOAc 75:25) to give 1.164 g of yellow oil, which after trituration in Petroleum Ether and filtration afforded 54a as a yellow solid (939 mg, 68% yield).

d) 2-bromo-1-(5-(2-(cyclopentyloxy)ethoxy)benzofuran-2-yl)ethanone 55a:

Under N₂, to a solution of 54a (558 mg, 1.935 mmol) in dry THF (50 mL) was added trimethylphenylammonium tribromide (763 mg, 2.032 mmol) over 40 minutes. The suspension was stirred 3 hours more at room temperature, quenched with 30 mL of water, and the THF is removed under reduced pressure. The aqueous phase was extracted with 3×30 mL of EtOAc, the combined organic phases rinsed twice with 10 mL of brine, dried over Ma₂SO₄, and filtered. After concentration of the filtrate, purification was achieved via FCC (eluent: Petroleum Ether-DCM 25:75) ) to give 704 mg of yellow solid, which after trituration in Petroleum Ether and filtration afforded 55a as a yellow solid (533 mg, 75% yield).

e) tert-butyl 2-(3-(4-(benzyloxy)phenyl)ureido)ethylcarbamate 58a:

4-benzyloxyphenylisocyanate 57a (commercially available, 25.5 g, 0.113 mol) was dissolved in DCM (700 mL), cooled to 0° C., and tert-butyl-2-aminoethylcarbamate 56 (commercially available, 19.045 g, 0.119 mol) was added dropwise over 30 minutes. The syrupy suspension was then stirred overnight at room temperature, resulting in the formation of a white precipitate, which is filtered, rinsed once with DCM and dried to afford 38.8 g of 58a as white solid (89% yield).

f) 1-(2-aminoethyl)-3-(4-(benzyloxy)phenyl)urea hydrochloride 59a:

58a (9.0 g, 0.023 mol) was suspended in 50 mL MeOH, and 150 mL of 4M HCl in dioxane was added to the stirred mixture: first dissolution occurs, and then precipitation. The suspension was stirred during 80 minutes, the gummy precipitate filtered, washed twice with Et₂O and dried under high vacuum to afford 6.38 g (85% yield) of 1-(2-aminoethyl)-3-(4-(benzyloxy)phenyl)urea hydrochloride 59a as a white solid. The filtrate was rotovaped until a small volume, extended with 100 mL of Et₂O and put on the fridge overnight to afford, after filtration, an additional 1.185 g (15% yield) of 59a as a white solid.

g) 1-(2-(benzylamino)ethyl)-3-(4-(benzyloxy)phenyl)urea 60a:

To a suspension of 59a (984 mg, 3.057 mmol) and benzaldehyde (340 mg, 3.202 mmol) in MeOH (40 mL) was added TEA (0.937 mL, 6.725 mmol), resulting in the dissolution of the suspension and then re-precipitation. After 30 minutes, NaBH₄ (177 mg, 4.680 mmol) was added to the mixture; 10 minutes later, the bubbling stopped and the resulting solution was quenched with 5 mL of water. The methanol was evaporated and the aqueous layer extracted with 3×30 mL of EtOAc. The combined organic layers were rinsed twice with 10 mL of brine, dried over Na₂SO₄, filtered, and the solvent evaporated to afford 1.068 g (93% yield) of 60a as a white powder.

h) 1-(2-(benzyl(2-(5-(2-(cyclopentyloxy)ethoxy)benzofuran-2-y)-2-oxoethyl)amino)ethyl)-3-(4-(benzyloxy)phenyl)urea 61a:

A suspension of 55a (459 mg, 1.25 mmol), the 1-(2-(benzylamino)ethyl)-3-(4-(benzyloxy)phenyl)urea 60a (469 mg, 1.25 mmol), and K₂CO₃ in a MeCN/DCM solution (40 mL+5 mL) was stirred at room temperature during 2 h 30 and then filtered. The solvent was evaporated and the residue was redissolved in DCM (50 mL), washed twice with 10 mL of water and twice with 10 mL of brine, dried over Na₂SO₄ and filtered. After concentration of the filtrate, purification was achieved via FCC (eluent: DCM-MeOH 93:7) to afford 674 mg (81% yield) of 61a as a yellow foam.

i) 1-(2-(2-(5-(2-(cyclopentyloxy)ethoxy)-2,3-dihydrobenzofuran-2-yl)-2-hydroxyethylamino)ethyl)-3-(4-hydroxyphenyl)urea 63a:

To a solution of 61a (100 mg, 0.151 mmol) in 20 mL of a mixture MeOH-H₂O-AcOH (7:2:1) was added 20 mg of 10% Pd/C, and the mixture was stirred under a hydrogen atmosphere (1.5 bar) during 4 days. The mixture was then filtered through a Celite plug using methanol. After concentration of the filtrate, purification was achieved via FCC (eluent: DCM-1M NH₃ in MeOH 90:10) to afford 12 mg of 63a as a beige solid.

j) 1-(2-(benzyl(2-(5-(2-(cyclopentyloxy)ethoxy)benzofuran-2-yl)-2-hydroxyethyl)amino)ethyl)-3-(4-(benzyloxy)phenyl)urea 62b:

To a solution of 61a (284 mg, 0.429 mmol) in MeOH (40 mL) at 0-5° C. is added NaBH₄ (23.7 mg, 0.627 mmol), and the mixture was stirred during 10 minutes before quenching with 10 mL of water. The MeOH was evaporated and the aqueous layer extracted with 3×50 mL of DCM, the combined organic layers washed with 10 mL of water and 10 mL of brine, dried over Na₂SO₄, and filtered. After concentration of the filtrate, purification was achieved via FCC (eluent: DCM-MeOH 90:10) to afford 230 mg (81% yield) of 62b as a yellow solid.

k) 1-(2-(2-(5-(2-(cyclopentyloxy)ethoxy)benzofuran-2-yl)-2-hydroxyethylamino)ethyl)-3-(4-hydroxyphenyl)urea 63b:

To a solution of 62b (112 mg, 0.169 mmol) in THF (10 mL) was added 0.5 mL of CHCl₃ and 15 mg of 10% Pd/C. The suspension was stirred under a hydrogen atmosphere (1 bar) during 24 h. Due to an incomplete conversion, a suspension of 20 mg of 10% Pd/C in 2 mL THF was added to the mixture, and the stirring followed for 24 h more. The suspension was then filtered through a Celite plug using a mixture DCM-1M NH₃ in MeOH. After concentration of the filtrate, purification was achieved via FCC (eluent: DCM-1M NH₃ in MeOH 90:10) to afford 33 mg of 63b as an off white solid.

By analogy to the preparation of 63b, compounds 63c and 63d were prepared from the corresponding 2-hydroxybenzaldehyde (4-methyl 52c and 5-methoxy 52d) following steps b) (first part to first evaporation, FCC (eluent: PE-DCM 95:5)), d), h) (60a+55c or 55d), j) and k) above.

### EXAMPLE 6

### Synthesis of Substituted benzofuran/dihydrobenzofuran Analogues (Scheme 8)

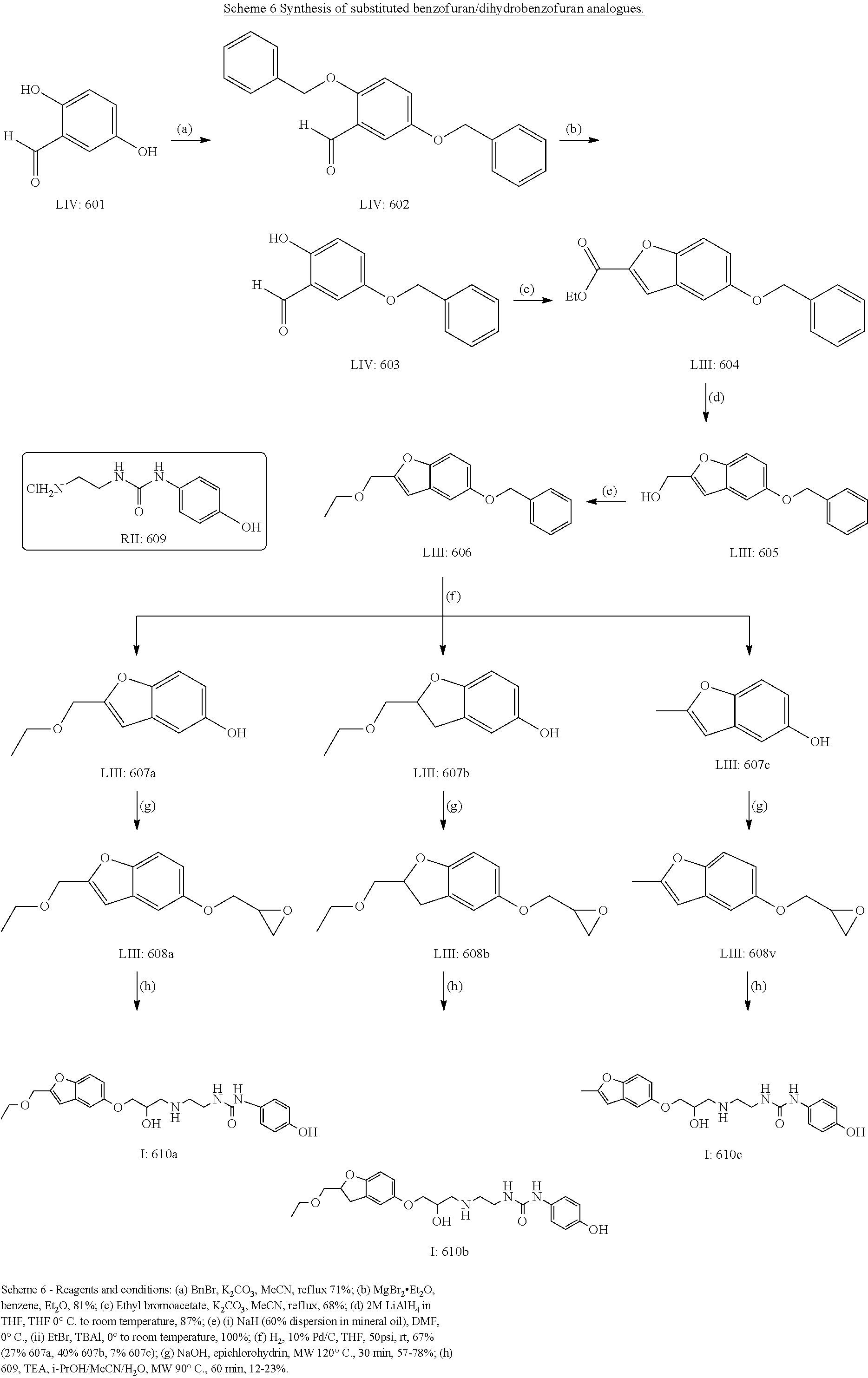

a) 2,5-bis(Benzyloxy)benzaldehyde (602)

2,5-Dihydroxybenzaldehyde 601 (3.267 g, 23.65 mmol) and potassium carbonate (7.192 g, 52.04 mmol, 2.2 eq) were suspended in MeCN (40 mL), with stirring. BnBr (4.045 g, 2.813 mL, 23.65 mmol, 1 eq) was added and then mixture heated under reflux for 1.5 hours. TLC analysis (eluent EtOAc/petroleum ether 40-60 3:7) showed the presence of starting aldehyde, so a further 0.1 eq of BnBr was added and the mixture stirred at 80° C. overnight. A further 0.1 eq of BnBr was added after overnight stirring. And after a further 1 hour of heating under reflux a final 0.1 eq of BnBr was added. Further heating under reflux for an hour was allowed before cooling and removal of solvent under reduced pressure to give a crude black solid. Attempts to partition the crude mixture between DCM (30 mL) and aq 1M HCl (30 mL) caused formation of a black emulsion. Attempts to break the emulsion by adding excess organic solvent were unsuccessful, so the mixture was shaken with celite and filtered, allowing separation of the layers. Concentration and purification of the crude residue by FCC (eluent EtOAc/PE 30:70) afforded 5.34 g (71%) of bis-protected 602 as a yellow crystalline solid.

b) 5-(Benzyloxy)-2-hydroxybenzaldehyde (603)

602 (3.70 g, 11.62 mmol) was suspended in benzene/Et₂O (7:1, 72 mL) under a nitrogen atmosphere. On addition of MgBr₂:Et₂O (3.30 g, 12.78 mmol, 1.1 eq), the solution turned from orange to black in colour. The mixture was heated under reflux overnight, before cooling and pouring onto aq. 2M HCl (50 mL). This mixture was then extracted with EtOAc (2×50 mL) and the combined organic layers concentrated. The crude mixture was purified by FCC (eluent EtOAc/PE 0.5:99.5 to 25:70 over 12 CV, then 25:75 to 50:50 over 3 CV) to give 2.15 g (81%) of pale yellow solid 603.

c) Ethyl 5-(benzyloxy)benzofuran-2-carboxylate (604)

603 (1.00 g, 4.38 mmol), ethyl bromoacetate (805 mg, 0.534 mL, 4.82 mmol, 1.1 eq) and potassium carbonate (1.817 g, 13.14 mmol, 3 eq) were dispersed in MeCN (35 mL) to give a yellow suspension. This was heated under reflux for 60 hours. Once cooled, the reaction mixture was poured onto water (100 mL) before extracting the suspension with EtOAc (3×30 mL). The combined organic layers were concentrated to give 1.033 g of yellow oil. This was further purified by FCC (eluent EtOAc/PE 10:90) to give 888 mg (68%) of white waxy solid 604.

d) (5-(Benzyloxy)benzofuran-2-yl)methanol (605)

LiAlH₄ 2M solution in THF (16.43 mL, 32.86 mmol, 1 eq) was diluted with dry THF (50 mL) under a nitrogen atmosphere at 0° C. A solution of 604 (9.737 g, 32.86 mmol) in dry THF (450 mL) was added slowly, before allowing the mixture to stir at room temperature for 30 minutes. The reaction mixture was cooled over ice, and quenched using the Fieser workup: addition of water (1.5 mL) followed aq. 2M NaOH (3 mL), followed water (3 mL). This caused formation of a white suspension, and the entire mixture was then passed through a bed of celite. The filtrate was concentrated to give pale yellow oil under reduced pressure. This solidified on standing overnight to a crystalline solid. The solid was redissolved in DCM (50 mL) and washed with water (30 mL) before drying to give 7.257 g (87%) of pale yellow crystalline solid. The product gave a single spot by TLC analysis (eluent EtOAc/PE 3:7, Rf=0.23) and ¹H-NMR analysis was clean, so the material 605 was used further without purification.

e) 5-(Benzyloxy)-2-(ethoxymethyl)benzofuran (606)

605 (4.00 g, 15.73 mmol) and NaH 60%s dispersion in mineral oil (750 mg, 18.88 mmol, 1.2 eq, equivalent to 453 mg of NaH) were dissolved in dry DMF (30 mL) at 0° C. under an atmosphere of nitrogen. EtBr (3.428 g, 2.348 mL, 31.46 mmol, 2 eq) and TBAI (1.162 g, 3.15 mmol, 0.2 eq) were then added and the reaction stirred at room temperature overnight. The mixture was quenched with water (5 mL), before removal of all volatiles under reduced pressure. The crude residue was dispersed in water (50 mL), then extracted with Et₂O (3×30 mL). The combined organic layers were washed with brine (20 mL). Concentration of the organic layers gave 4.47 g (100%) of yellow oil 606, which was used without further purification.

f) 2-(Ethoxymethyl)benzofuran-5-ol (607a), 2-(ethoxymethyl)-2,3-dihydrobenzofuran-5-ol (607b) and 5-hydroxy-2-methylbenzofuran (607c)

606 (2.00 g, 7.01 mmol) was dissolved in THF (20 mL) in a Parr-hydrogenation vessel. 10% Pd/C (250 mg) was added with care, first ensuring an inert atmosphere of nitrogen was present in the vessel. A further 5 mL of THF were added to rinse down the vessel walls, before sonicating the mixture for 5 minutes. The mixture was hydrogenated under 50 psi of pressure at room temperature, overnight. TLC analysis (eluent EtOAc/PE 3:7) indicated total disappearance of the SM, but formation of 3 new spots, with lower Rf values. The reaction mixture was filtered over a bed of celite, with washings of MeOH, before concentration of the filtrate to give 1.336 g of crude product as a brown oil. This was purified by FCC (eluent EtOAc/PE 1:99 to 10:90 over 5 CV, hold at 10:90 for 5 CV, then to 40:60 over 10 CV) to give 607c as a yellow oil, 69 (7%).

(EtOAc/PE 1:99 to 10:90 over 5 CV, 10:90 for 5 CV, to 20:80 over 5 CV, then 20:80 till complete elution) to give 607a, 362 mg (27%), 607b, 549 mg (40%).

g) 2-(Ethoxymethyl)-5-(oxiran-2-ylmethoxy)benzofuran (608a)

607a (350 mg, 1.82 mmol), NaOH (76 mg, 1.91 mmol, 1.05 eq) and epichlorohydrin (1 mL) were placed in a 10 mL MW reaction vessel and heated at 120° C. for 30 minutes (dynamic program, max pressure 250 psi, max power 300 W). The reaction mixture was dispersed in water (20 mL), before washing with DCM (3×10 mL) and concentrating the combined organic layers under reduced pressure to give 451 mg of crude product. This was further purified by FCC (eluent EtOAc/PE 0:100 to 60:40 over 10 CV, with a plateau at 10:90 for 5 CV (total run 15 CV) to give 353 mg (78%) of clear colourless oil 608a.

2-(Ethoxymethyl)-5-(oxiran-2-ylmethoxy)-2,3-dihydrobenzofuran (608b)

607b was alkylated in a similar manner to 607a as described in the synthesis of 608a, 608b obtained. Yield: 71%.

2-Methyl-5-(oxiran-2-ylmethoxy)benzofuran (608c)

607c was alkylated in a similar manner to 607a as described in the synthesis of 608a, 608c obtained. Yield: 57%.

h) 1-(2-(3-(2-(Ethoxymethyl)benzofuran-5-yloxy)-2-hydroxypropylamino)ethyl)-3-(4-hydroxyphenyl)urea (610a)

Synthesis of 609 is described in UK Patent Application No 0911657.5.

608a (108 mg, 0.44 mmol) and 609 (204 mg, 0.88 mmol, 2 eq) were dispersed in propan-2-ol/MeCN/water (7:2:1, 3 mL), before adding TEA (129 μL, 0.92 mmol, 2.1 eq). The resulting solution was heated at 90° C. in the MW reactor on a dynamic program (max pressure=250 psi, max power=300 W) for 1 hour. The reaction mixture was concentrated under reduced pressure, and the crude product purified by FCC (eluent 1N NH₃ in MeOH/DCM 0.100 for 2 CV to prime, 0:100 to 5:95 over 5 CV, then hold at 5:95 for 5 CV, to 10:90 over 5 CV, then hold at 10:90 for 5 CV, to 15:85 over 5 CV) to give 45 mg of a white solid 610a

1-(2-(3-(2-(Ethoxymethyl-2,3-dihydrobenzofuran-5-yloxy-2-hydroxypropylamino)ethyl)-3-(4-hydroxyphenyl)urea (610b)

608b underwent aminolysis with 609 according to the procedure described for the synthesis of 610a, 610b obtained.

1-(2-(2-Hydroxy-3-(2-methylbenzofuran-5-yloxy)propylamino)ethyl)-3-(4-hydroxyphenyl)urea (610c)

608c underwent aminolysis with 609 according to the procedure described for the synthesis of 610a, 610c obtained.

### EXAMPLE 7

### Synthesis of Substituted benzodioxan Analogues (Scheme 7)

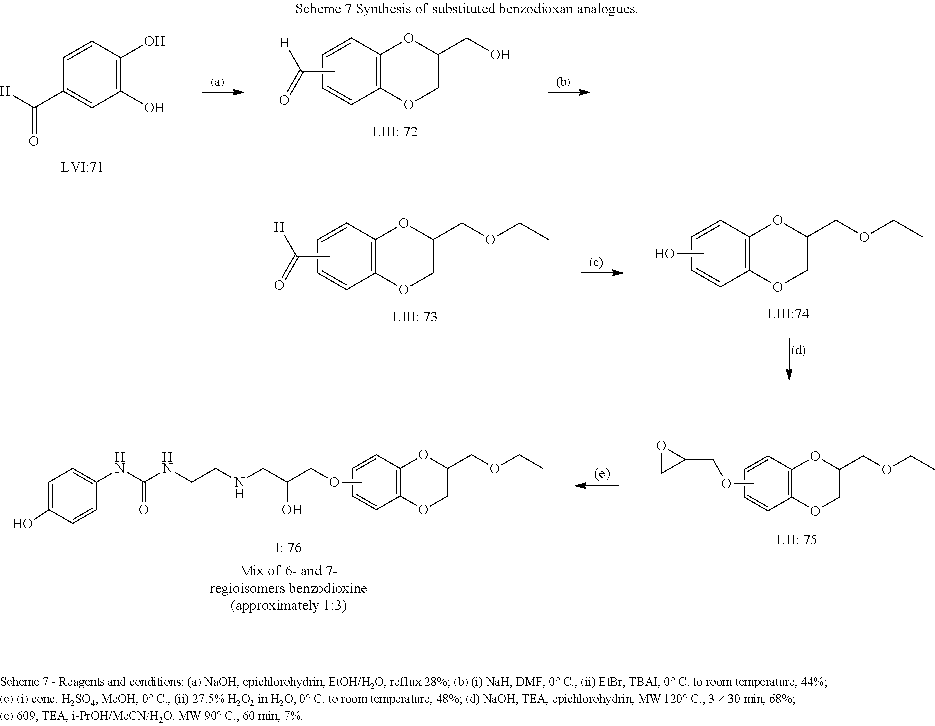

a) 3-(Hydroxymethyl)-2,3-dihydrobenzo[b][1,4]dioxine-6-carbaldehyde and 2-(hydroxymethyl)-2,3-dihydrobenzo[b][1,4]dioxine-6-carbaldehyde (72)

3,4-Dihydroxybenzaldehyde 71 (5.00 g, 36.20 mmol) was dissolved in a solution of EtOH/H₂O (5.3, 200 mL) containing NaOH (3.04 g, 76.02 mmol, 2.1 eq) and epichlorohydrin (3.68 g, 3.11 mL, 39.82 mmol, 1.1 eq), at room temperature. The mixture was heated at 75° C. for 2 days. After removal of EtOH under reduced pressure, an additional 50 mL of water was added and the mixture extracted with DCM (3×30 mL, taking the emulsion into the organic phase). The combined organic phases were washed with brine (40 mL) before concentration under reduced pressure to give a dark oil. This was further purified by FCC (eluent EtOAc/PE 12.5:87.5 to 100% EtOAc over 10 CV) to give 1.965 g (28%) of orange solid (desired products present as a mixture of regioisomers, 3-(hydroxymethyl)-2,3-dihydrobenzo[b][1,4]dioxine-6-carbaldehyde 72 present as the major isomer at around 75%)

b) 3-(Ethoxymethyl)-2,3-dihydrobenzo[b][1,4]dioxine-6-carbaldehyde and 2-(ethoxymethyl)-2,3-dihydrobenzo[b][1,4]dioxine-6-carbaldehyde (73)

The regioisomeric mix of 72 (1.930 g, 9.94 mmol) and NaH 60% dispersion in oil (477 mg, 11.93 mmol, 1.2 eq, equivalent to 286 mg of NaH) were dispersed in dry DMF at 0° C. under an atmosphere of nitrogen, giving a brown suspension. EtBr (1.484 mL, 19.88 mmol, 2 eq) and TBAI (734 mg, 1.099 mmol, 0.2 eq) were added, and the reaction stirred at room temperature overnight, with the mixture turning clear orange as the reaction progressed. Water (5 mL) was added to quench the reaction, before concentrating the reaction mixture under reduced pressure. The residue was dispersed in water (30 mL) before extracting with diethyl ether (3×30 mL), NaCl was used to saturate the aqueous layer before the final organic wash. The combined organic layers were then washed with brine (20 mL) to give 2.014 g of amber oil as the crude product. This was further purified by FCC (eluent EtOAc/PE 1:99 to 10:90 over 5 CV, hold 5 CV to 40:60 over 12 CV) to give 964 mg (44%) of a yellow oil. The title compounds 73 were present in the same ratio as the starting materials.

c) 3-(Ethoxymethyl)-2,3-dihydrobenzo[b][1,4]dioxin-6ol and 2-(ethoxymethyl)-2,3-dihydrobenzo[b][1,4]dioxin-6-ol (74)

The regioisomeric mix of 73 (794 mg, 3.57 mmol) was dissolved in a mixture of MeOH/cH₂SO₄ (100:1, 10 mL) and cooled to 0° C. to produce a yellow solution. H₂O₂ 27.5% in water (575 μL, 4.64 mmol, 1.3 eq) was added at 0° C., before allowing the mixture to warm to room temperature and stirring continued overnight. TLC analysis (eluent EtOAc/PE 30:70) indicated the starting aldehydes were still present, so a further 0.2 eq of H₂O₂ were added and the mixture stirred at room temperature overnight once more. All volatiles were removed under reduced pressure, before dispersing the residue in water (20 mL) and extracting with DCM (3×20 mL). The combined organic extracts were dried and concentrated to give red/brown oil (779 mg). This was purified further by FCC (eluent EtOAc/PE 2:98 to 50:50 over 10 CV, with plateau at 25% for 3 CV) the wavelength was kept at 220 nm due to poor absorbance at 254 nm. The regioisomeric mix of desired products 74 was obtained as 360 mg (48%) of red oil in with the ratio intact.

d) 2-(Ethoxymethyl)-7-(oxiran-2-ylmethoxy)-2,3-dihydrobenzo[b][1,4]dioxine and 2-(ethoxymethyl)-6-(oxiran-2-ylmethoxy)-2,3-dihydrobenzo[b][1,4]dioxine (75)

The regioisomeric mix of 74 (310 mg, 1.47 mmol), NaOH (62 mg, 1.55 mmol, 1.05 eq) and epichlorohydrin (2 mL) were heated at 120° C. in the MW reactor for 2×30 minute cycles (dynamic 300 W, 250 psi). TLC (eluent 100% DCM) and LCMS analysis indicated a large amount of starting material was still present, so triethylamine (0.205 mL, 1 eq) was added and a further cycle of heating carried out, after which time all starting material had disappeared. The reaction mixture was diluted with water (20 mL), before extraction with DCM (3×20 mL). The combined organic layers were washed with 0.5M HCl (aq) (20 mL) before concentrating. The crude residue was further purified by FCC (eluent DCM/PE 0:100 to 100:0 over 12 CV, with plateaus for 3 CV at 0:33 and 0:67 respectively), to give 247 mg (63%), of clear yellow oil 75 comprising of both the 6- and 7-substituted benzodioxine regioisomers (1:3), without resolution of the individual diastereoisomers.

e) 1-(2-(3-(3-(Ethoxymethyl)-2,3-dihydrobenzo[b][1,4]dioxin-6-yloxy)-2-hydroxypropylamino)ethyl)-3-(4-hydroxyphenyl)urea and 1-(2-(3-(2-(ethoxymethyl)-2,3-dihydrobenzo[b][1,4]dioxin-6-yloxyl-2-hydroxypropylamino)ethyl)-3-(4-hydroxyphenyl)urea (76)

The regioisomeric mix of 75 underwent aminolysis with 609 according to the procedure described for the synthesis of 610a. Product 76 was obtained.

Analytical data for the product compounds is shown in Data Table 1

| Ex No | Yield | m.p/C | LC MS/(M + 1)/z |
|---|---|---|---|
| 4a | | N/A | C18: 2′21/510.2/512.0 Luna: 2′42 |
| 4b | | 165 | C18: 2′20/508.1/510.1 Luna: 2′41 |
| 4c | | 160 | C18: 1′96/432.0/434.1 Luna: 2′21 |
| 4d | | 158 | C18: 2′01/446.0/448.0 Luna: 2′24 |
| 24a | 12 | N/A | C18: 2′14/477.4/478.3/479.1 Luna: 2′39 |
| 24b | | N/A | C18: 1′88/447.1/449.1 Luna: 2′09 |
| 46a | 42 | 141 | C18: 2′08/392.9/394.9 Luna: 2′31 |
| 46b | 48 | 141 | C18: 0′85/374.5 Luna: 1′90 |
| 63a | 16 | N/A | C18: 2′05/486.7/488.1 Luna: 2′24 |
| 63b | 40 | N/A | C18: 2′09/484.0/485.1 Luna: 2′27 |
| 63c | 53 | 152.5 | C18: 1′85/370.3; Luna: 2′07 |
| 63d | 51 | 151 | C18: 1′14/386.2; Luna: 1′95 |
| 76 | 7 | N/A | C18: 1′80/463.0 Luna: 2′05 |
| 610a | 23 | 131.5 | C18: 1′89/444.5 Luna: 2′13 |
| 610b | 23 | 133 | C18: 1′89/400.1 Luna: 2′16 |
| 610c | 12 | 138 | C18: 1′68/447.0 Luna: 2′00 |

### EXAMPLE A1

### Ligand Binding Studies

Selectivity of ligands for the three beta-adrenoceptors was assessed by whole-cell binding studies using ³H-CGP12177 in CHO cells expressing the human beta1, beta2 or beta3-adrenoceptors respectively essentially as described by Baker (2005; Br. J Pharmacol: 144, 317-22). Values shown are K_{D} values determined as described by Baker (2005). The K_{D} values for each ligand at the human beta1, beta2 and beta3 adrenoceptors are shown in Table 2. K_{D} represents the concentration of compound required to occupy 50% of the receptors in cells or tissues.

The selectivity of a ligand is given by the ratio of beta-1 to beta-2 K_{D}. Accordingly a difference of one in the logarithmic values thereof represents a 10-fold selectivity, a difference of 2 represents 100-fold selectivity and a difference of 3 represents 1000-fold selectivity etc.

TABLE 2
3H-CGP 12177 Whole cell binding

| | Beta 1 | Beta 2 | Beta 1 − Beta 2 |
|---|---|---|---|
| Ex No | Log K_{D} | Log K_{D} | Log K_{D} |
| 4a | −6.39 | −5.51 | 0.88 |
| 4b | −6.21 | −5.35 | 0.86 |
| 4c | −6.45 | −5.03 | 1.42 |
| 4d | −6.41 | −5.35 | 1.06 |
| 24a | −6.50 | −5.27 | 1.24 |
| 24b | −6.30 | −5.19 | 1.10 |
| 46a | −8.40 | −7.24 | 1.16 |
| 46b | −7.96 | −7.01 | 0.95 |
| 63a | −6.27 | −4.57 | 1.70 |
| 63b | −6.81 | −4.35 | 2.45 |
| 63c | −7.33 | −6.28 | 1.06 |
| 63d | −6.65 | −5.50 | 1.14 |
| 76 | −6.84 | −4.69 | 2.15 |
| 610a | −6.59 | −4.99 | 1.60 |
| 610b | −6.78 | −4.79 | 1.99 |
| 610c | −6.55 | −5.16 | 1.39 |

1. A compound of formula I, and its pharmaceutical acceptable salt or salts and physiologically hydrolysable derivatives in free form or salt form: 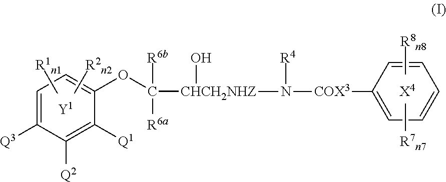
wherein
either Q¹, CR^{6a} and optionally R^{6b} together form a cyclic moiety wherein:
Q¹ is selected from C₁₋₂ alkylene, C₁₋₂ alkenylene, OC₁ alkylene and OC₁ alkenylene moieties optionally substituted by oxo;
R^{6a} is a single bond and R^{6b} is H; or
R^{6a} and R^{6b} together form a double bond; and
Q² and Q³ are independently selected from H, R¹ and R²;
or Q² and Q³ together form a cyclic moiety in which one of Q² and Q³ is a cyclic moiety selected from OC₁ alkylene and OC₁ alkenylene moieties optionally substituted by oxo or a group R⁵ as hereinbelow defined for R² and the other of Q² and Q³ is a cyclic moiety selected from alkylene, C₁₋₂ alkenylene and OC₁ alkylene optionally substituted by oxo;
R^{6a} and R^{6b} are each H or a cyclic moiety as defined above; and
Q¹ is selected from H, R¹ and R² and a cyclic moiety as defined above;
and R¹ is independently selected from F, Cl, Br, CN, NH₂, OH, CHO, COOH, CONH₂ and SO₂NH₂,
R² is independently selected from NHR³, NO₂, CF₃, OR³, COR³, OCOR³, COOR³, CONR³₂, NR³COR³, CONR³₂, SO₂NR³₂, NR³SO₂R³, C₁₋₅alkyl, C₁₋₅alkoxy, C₂₋₅alkenyl, C₂₋₅alkynyl, -W-C₃₋₁₀cycloalkyl and -W-C₅₋₁₀carbocyclyl wherein W is C₁₋₅alkylene, C₂₋₅alkenylene or C₂₋₅alkynylene;
any of which may comprise one or more carbonyl units or heteroatoms selected from O, S and N and which may be unsubstituted or further substituted by one of more R²¹;
R²¹ is independently selected from C₁₋₅alkyl, C₂₋₅alkenyl, C₂₋₅alkynyl and a group as defined for R¹
wherein the C₁₋₅alkyl, C₂₋₅alkenyl, C₂₋₅alkynyl groups are optionally substituted by one or more groups independently selected from R¹;
R³ is independently selected from C₁₋₅alkyl, C₁₋₅alkoxy, C₂₋₅alkenyl, C₂₋₅alkynyl, aryl, C₃₋₁₀cycloalkyl, and C₅₋₁₀carbocyclyl;
any of which may comprise one or more carbonyl units or heteroatoms selected from O, S and N and which may be unsubstituted or further substituted by one of more groups independently selected from R¹;
R⁴ is selected from H and C₁₋₅alkyl;
n1 and n2 and the sum thereof are independently selected from zero and a whole number integer 1 and 2;
Z is selected from linear C₂₋₃ alkylene;
X³ is NH;
R⁷ is independently selected from F, Cl, Br, CN, NH₂, OH, CHO, COOH, CONH₂ and SO₂NH₂,
R⁸ is independently selected from NHR⁹, NO₂, CF₃, OR⁹, COR⁹, OCOR⁹, COOR⁹, COONR⁹₂, NR⁹COR⁹, CONR⁹₂, SO₂NR⁹₂, NR⁹SO₂R⁹, C₁₋₅alkyl, C₁₋₅alkoxy, C₂₋₅alkenyl, C₂₋₅alkynyl, -Z²-C₃₋₁₀cycloalkyl and -Z²-C₅₋₁₀carbocyclyl wherein Z² is C₁₋₅alkylene, C₂₋₅alkenylene or C₂₋₅alkynylene;
any of which may comprise one or more carbonyl units or heteroatoms selected from O, S and N and which may be unsubstituted or further substituted by one of more R⁸¹;
R⁸¹ is independently selected from C₁₋₅alkyl, C₂₋₅alkenyl, C₂₋₅alkynyl and a group as defined for R⁷
wherein the C₁₋₅alkyl, C₂₋₅alkenyl, C₂₋₅alkynyl groups are optionally substituted by one or more groups independently selected from R⁷;
R⁹ is independently selected from C₁₋₅alkyl, C₁₋₅alkoxy, C₂₋₅alkenyl, C₂₋₅alkynyl, aryl, C₃₋₁₀cycloalkyl, and C₅₋₁₀carbocyclyl;
any of which may comprise one or more carbonyl units or heteroatoms selected from O, S and N and which may be unsubstituted or further substituted by one of more groups independently selected from R⁷; and
n7 and n8 and the sum thereof are independently selected from zero and the whole number integer 1 to 4.

2. A compound as claimed in claim 1 which comprises an optionally substituted bicyclic heteroaromatic ring system comprising benzene ring Y¹ fused to a heterocyclic or heteroaromatic 5 or 6 membered ring, comprising at least one O-heteroatom and optionally including an oxo (═O) moiety, wherein the linking atom for the ring system to the remainder of the compound is a C atom of either ring, and wherein the ring system optionally incorporates the OCR^{6a}R^{6b} moiety.

3. A compound as claimed in claim 1, being of subformula Ia, and its pharmaceutically acceptable salt or salts and physiologically hydrolysable derivatives: 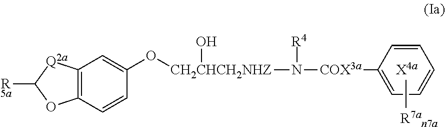
wherein Q^{2a} is selected from COCH₂, COCH, CHCH, CH₂, CH and OCH₂;
R^{5a} is selected from Ph, oxo, alkyl, alkoxyalkyl, CO₂NH₂ and CO₂alkyl;
X^{3a} is NH;
R^{7a} is 3-Cl or 4-OH; and
n7a is 1,
wherein all other integers are as defined in claim 1.

4. A compound as claimed in claim 1, being of subformula Ib, and its pharmaceutically acceptable salt or salts and physiologically hydrolysable derivatives: 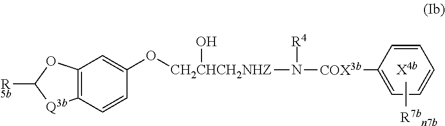
wherein Q^{3b} is selected from COCH₂ and OCH₂;
R^{5b} is selected from Ph and CH₂OC₂H₅;
X^{3b} is NH;
R^{7b} is 3-Cl or 4-OH; and
n7b is 1; and
wherein all other integers are as defined in claim 1.

5. A compound as claimed in claim 1, being of subformula Ic, and its pharmaceutically acceptable salt or salts and physiologically hydrolysable derivatives: 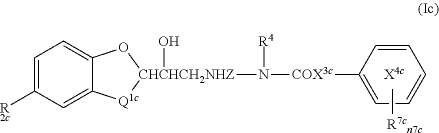
wherein Q^{1c} is selected from CH₂CH₂ CH₂, CH₂CH, CH, CHCH, OCH₂, COCH, and COCH;
R^{2c} is selected from H and cycloalkoxyalkoxyl;
X^{3c} is NH;
R^{7c} is 3-Cl or 4-OH; and
n7c is 1;
wherein all other integers are as defined in claim 1.

6. A compound of formula I as claimed in claim 1 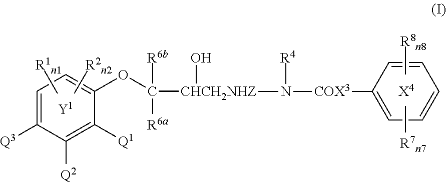
as given in the following Table 1

| Ex. | R⁵ | R¹, R² | Q²Q3Y¹Q¹OCR^{6a,6b} | Z | X³ | R⁷, R⁸ |
|---|---|---|---|---|---|---|
| 4a | 2-Ph | — | 7-Cha4O—OCH2 | (CH₂)₂ | NH | 3-Cl |
| 4b | 2-Ph | — | 6-Che4O—OCH2 | (CH₂)₂ | NH | 3-Cl |
| 4c | — | — | 6-Che2O—OCH2 | (CH₂)₂ | NH | 3-Cl |
| 4d | — | — | 6-Che2O-(4-Me)—OCH2 | (CH₂)₂ | NH | 3-Cl |
| 24a | 2-CO₂C₂H₅ | — | 5-BzF—OCH2 | (CH₂)₂ | NH | 3-Cl |
| 24b | 2-CO₂NH₂ | — | 5-BzF—OCH2 | (CH₂)₂ | NH | 3-Cl |
| 46a | — | — | 2-BzDx | (CH₂)₂ | NH | 3-Cl |
| 46b | — | — | 2-BzDx | (CH₂)₂ | NH | 4-OH |
| 63a | — | 5-O(CH₂)₂Oc • pent | 2-DhBzF | (CH₂)₂ | NH | 4-OH |
| 63b | — | 5-O(CH₂)₂Oc • pent | 2-BzF | (CH₂)₂ | NH | 4-OH |
| 63c | — | 6-CH₃ | 2-BzF | (CH₂)₂ | NH | 4-OH |
| 63d | — | 5-OCH₃ | 2-BzF | (CH₂)₂ | NH | 4-OH |
| 76 | 2-CH₂OC₂H₅ | — | 7-BzDx—OCH₂ | (CH₂)₂ | NH | 4-OH |
| 610a | 2-CH₂OC₂H₅ | — | 5-BzF—OCH₂ | (CH₂)₂ | NH | 4-OH |
| 610b | 2-CH₂OC₂H₅ | — | 5-DhBzF—OCH₂ | (CH₂)₂ | NH | 4-OH |
| 610c | 2-CH₃ | — | 5-BzF—OCH₂ | (CH₂)₂ | NH | 4-OH |

wherein
Cha4O=Chroman-4-one Che4O=Chromen-4-oneChe2O=chromen-2-one 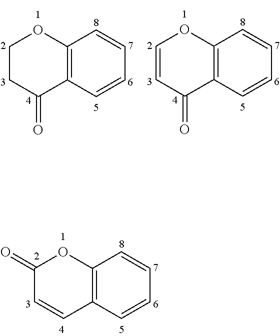
BzF=benzofuran DhBzF=dihydrobenzofuran BzDx=1,4-benzodioxane 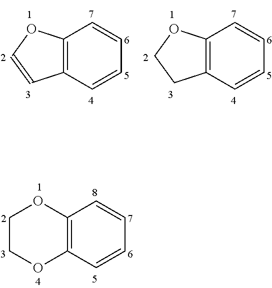
and wherein R⁴ is H and X⁴ is phenyl.

7. A process for preparing a compound of formula as defined in claim 1 comprising reacting a compound of formula LII or LV:
Q³Q²Y¹Q¹OCR^{6a}R^{6b} oxirane LII
Q³Q²Y¹Q¹OCR^{6a}R^{6b}CORCH₂R^{L} where R=a bond and R^{L}=Br LV
with a compound of formula RII:
H₂NZNR⁴ COX³X⁴.

8. Novel intermediates as defined in Claim 7.

9. A composition comprising a therapeutically effective amount of a compound of formula I or subformulae or its pharmaceutically acceptable salt and physiologically hydrolysable derivative as defined in claim 1 in association with one or more pharmaceutical carriers or diluents.

10.-14. (canceled)

15. A method of treating a condition selected from ischaemic heart disease (also known as myocardial infarction or angina), hypertension, and heart failure, restenosis and cardiomyopathy, said method comprising administering to a subject in need thereof, a composition comprising a compound of formula I or subformulae or pharmaceutically acceptable salt or composition thereof as defined in claim 1 in an amount sufficient to treat the condition.

16. The method of claim 15, wherein the subject has concomitant respiratory disease.

17. The method of claim 16, wherein the respiratory disease is selected from asthma and COPD.

18. The method claim 15, wherein the composition further comprises one or more pharmaceutical carriers or diluents.

19. A compound as claimed in claim 2, being of subformula Ia, and its pharmaceutically acceptable salt or salts and physiologically hydrolysable derivatives: 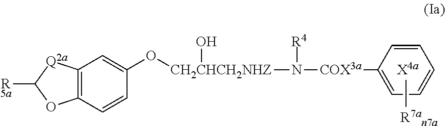
wherein Q^{2a} is selected from COCH₂, COCH, CHCH, CH₂, CH and OCH₂;
R^{6a} is selected from Ph, oxo, alkyl, alkoxyalkyl, CO₂NH₂ and CO₂alkyl;
X^{3a} is NH;
R^{7a} is 3-Cl or 4-OH; and
n7a is 1,
wherein all other integers are as defined in claim 2.

20. A compound as claimed in claim 2, being of subformula Ib, and its pharmaceutically acceptable salt or salts and physiologically hydrolysable derivatives: 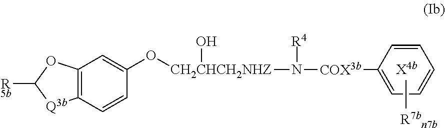
wherein Q^{3b} is selected from COCH₂ and OCH₂;
R^{5b} is selected from Ph and CH₂OC₂H₅;
X^{3b} is NH;
R^{7b} is 3-Cl or 4-OH; and
n7b is 1; and
wherein all other integers are as defined in claim 2.

21. A compound as claimed in claim 2, being of subformula Ic, and its pharmaceutically acceptable salt or salts and physiologically hydrolysable derivatives: 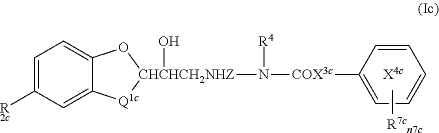
wherein Q^{1c} is selected from CH₂CH₂ CH₂, CH₂CH, CH, CHCH, OCH₂, COCH₂ and COCH;
R^{2c} is selected from H and cycloalkoxyalkoxyl;
X^{3c} is NH;
R^{7c} is 3-Cl or 4-OH; and
n7c is 1;
wherein all other integers are as defined in claim 2.

22. A compound of formula I as claimed in claim 2 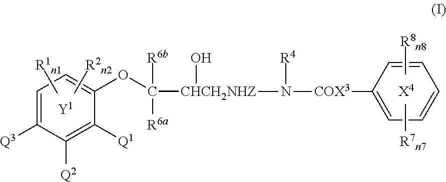
as given in the following Table 1

| Ex. | R⁵ | R¹, R² | Q²Q³Y¹Q¹OCR^{6a,6b} | Z | X³ | R⁷, R⁸ |
|---|---|---|---|---|---|---|
| 4a | 2-Ph | — | 7-Cha4O—OCH₂ | (CH₂)₂ | NH | 3-Cl |
| 4b | 2-Ph | — | 6-Che4O—OCH₂ | (CH₂)₂ | NH | 3-Cl |
| 4c | — | — | 6-Che2O—OCH₂ | (CH₂)₂ | NH | 3-Cl |
| 4d | — | — | 6-Che2O-(4-Me)—OCH₂ | (CH₂)₂ | NH | 3-Cl |
| 24a | 2-CO₂C₂H₅ | — | 5-BzF—OCH₂ | (CH₂)₂ | NH | 3-Cl |
| 24b | 2-CO₂NH₂ | — | 5-BzF—OCH₂ | (CH₂)₂ | NH | 3-Cl |
| 46a | — | — | 2-BzDx | (CH₂)₂ | NH | 3-Cl |
| 46b | — | — | 2-BzDx | (CH₂)₂ | NH | 4-OH |
| 63a | — | 5-O(CH₂)₂Oc.pent | 2-DhBzF | (CH₂)₂ | NH | 4-OH |
| 63b | — | 5-O(CH₂)₂Oc.pent | 2-BzF | (CH₂)₂ | NH | 4-OH |
| 63c | — | 6-CH₃ | 2-BzF | (CH₂)₂ | NH | 4-OH |
| 63d | — | 5-OCH₃ | 2-BzF | (CH₂)₂ | NH | 4-OH |
| 76 | 2-CH₂OC₂H₅ | — | 7-BzDx-OCH₂ | (CH₂)₂ | NH | 4-OH |
| 610a | 2-CH₂OC₂H₅ | — | 5-BzF—OCH₂ | (CH₂)₂ | NH | 4-OH |
| 610b | 2-CH₂OC₂H₅ | — | 5-DhBzF—OCH₂ | (CH₂)₂ | NH | 4-OH |
| 610c | 2-CH₃ | — | 5-BzF—OCH₂ | (CH₂)₂ | NH | 4-OH |

wherein
Cha4O=Chroman-4-one Che4O=Chromen-4-oneChe2O=chromen-2-one 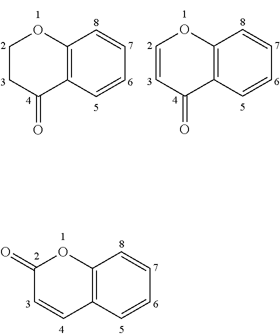
BzF=benzofuran DhBzF=dihydrobenzofuran BzDx=1,4-benzodioxane 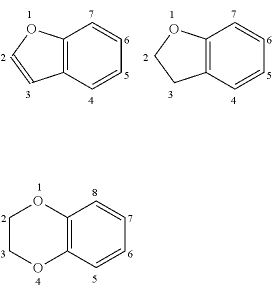
and wherein R⁴ is H and X⁴ is phenyl.
